(19) United States
(12) Patent Application Publication
(10) Pub. No.: US 2015/0166515 A1
(43) Pub. Date: Jun. 18, 2015

(54) MATERIAL FOR ORGANIC ELECTROLUMINESCENCE DEVICE AND ORGANIC ELECTROLUMINESCENCE DEVICE INCLUDING THE SAME

(71) Applicant: SAMSUNG DISPLAY CO., LTD., Yongin-City (KR)

(72) Inventor: Hiroaki ITOI, Yokohama (JP)

(21) Appl. No.: 14/573,937

(22) Filed: Dec. 17, 2014

(30) Foreign Application Priority Data
Dec. 18, 2013 (JP) 2013-261756

(51) Int. Cl. C07D 405/14; C07D 409/14; H01L 51/50; H01L 51/00

(52) U.S. Cl. CPC C07D 405/14; H01L 51/0072; H01L 51/0073; C07D 409/14; H01L 51/0074; H01L 51/0052; H01L 51/0058; H01L 51/0061; H01L 51/5012

(57) ABSTRACT

A compound for an organic EL device and an organic EL device, the compound being represented by the following Formula (1):

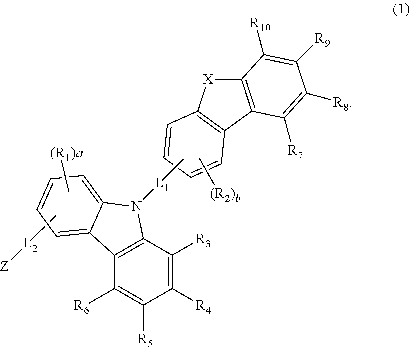

### CROSS-REFERENCE TO RELATED APPLICATION

Japanese Patent Application No. 2013-261756, filed on Dec. 18, 2013, in the Japanese Patent Office, and entitled: “Material for Organic Electroluminescence Device and Organic Electroluminescence Device Including the Same,” is incorporated by reference herein in its entirety.

### BACKGROUND

1. Field

Embodiments relate to a material for an organic electroluminescence device and an electroluminescence device including the same.

2. Description of the Related Art

In recent years, organic electroluminescence (EL) displays are one type of image displays that have been actively developed.

Unlike a liquid crystal display and the like, the organic EL display is a self-luminescent display that recombines holes and electrons injected from an anode and a cathode in a light emitting layer to thus emit lights from a light-emitting material including an organic compound of the light emitting layer, thereby performing display.

An example of an organic electroluminescence device (hereinafter referred to as an organic EL device) may include an organic EL device that includes an anode, a hole transport layer disposed on the anode, a light emitting layer disposed on the hole transport layer, an electron transport layer disposed on the light emitting layer, and a cathode disposed on the electron transport layer. Holes injected from the anode may be injected into the light emitting layer via the hole transport layer. Meanwhile, electrons may be injected from the cathode, and then injected into the light emitting layer via the electron transport layer. The holes and the electrons injected into the light emitting layer may be recombined to generate excitons within the light emitting layer. The organic EL device emits light by using lights generated by the transition of the excitons to a ground state.

### SUMMARY

Embodiments are directed to a material for an organic electroluminescence device and an electroluminescence device including the same.

The embodiments may be realized by providing a compound for an organic electroluminescence (EL) device, the compound being represented by the following Formula (1):

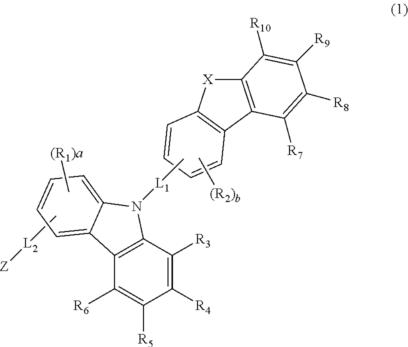

wherein, in Formula 1, X is an oxygen atom or a sulfur atom, R₁-R₁₀ are each independently an aryl group having 6 to 18 ring carbon atoms, a heteroaryl group having 5 to 18 ring carbon atoms, an alkyl group having 1 to 15 carbon atoms, a silyl group, a halogen atom, a hydrogen atom or a deuterium atom, a and b are each independently an integer of 0 to 3, L₁ is a divalent connecting group or a single bond, L₂ is a divalent connecting group, and Z is an aryl group having 6 to 30 ring carbon atoms or a heteroaryl group having 5 to 30 ring carbon atoms, wherein, when L₁ is the single bond, none of R₇-R₁₀ are a heteroaryl group.

At least two adjacent ones of R₁-R₁₀ may be combined to each other and form a saturated or unsaturated ring, provided that an aromatic ring is not formed by a combination of R₁ with R₃ and R₆, or R₂ with R₇ and R₁₀.

A dibenzofuranyl moiety or a dibenzothiophenyl moiety of Formula (1) may be bound to L₁ at position 2, position 3, or position 4 of the dibenzofuranyl moiety or the dibenzothiophenyl moiety.

The embodiments may be realized by providing a compound for an organic electroluminescence (EL) device, the compound being represented by the following Formula (2):

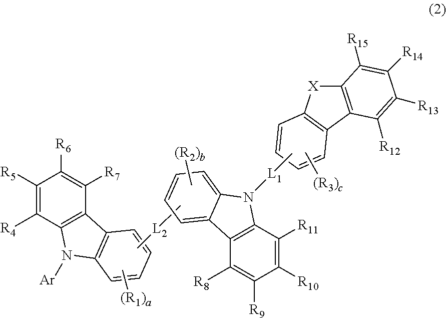

wherein, in Formula (2), X is an oxygen atom or a sulfur atom, Ar is an aryl group having 6 to 18 ring carbon atoms or an alkyl group having 1 to 15 carbon atoms, R₁-R₁₅ are each independently an aryl group having 6 to 30 ring carbon atoms, an alkyl group having 1 to 15 carbon atoms, a silyl group, a halogen atom, a hydrogen atom or a deuterium atom, a, b and c are each independently an integer of 0 to 3, L₁ is a divalent connecting group or a single bond, and L₂ is a divalent connecting group.

At least two adjacent ones of R₁-R₁₅ may be combined to each other and form a saturated or unsaturated ring, provided that an aromatic ring is not formed by a combination of R₁ with R₄ and R₇, R₂ with R₈ and R₁₁, or R₃ with R₁₂ and R₁₅.

A dibenzofuranyl moiety or a dibenzothiophenyl moiety of Formula (2) may be bound to L₁ at position 2, position 3, or position 4 of the dibenzofuranyl moiety or the dibenzothiophenyl moiety.

The embodiments may be realized by providing a compound for an organic electroluminescence (EL) device, the compound being represented by the following Formula (3):

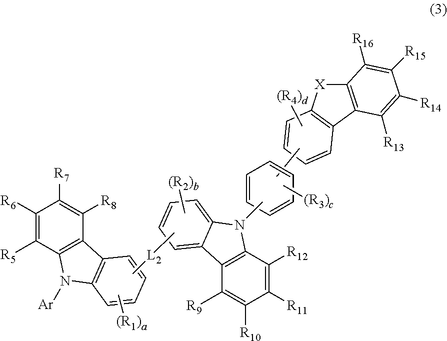

wherein, in Formula (3), X is an oxygen atom or a sulfur atom, Ar is an aryl group having 6 to 18 ring carbon atoms, a heteroaryl group having 5 to 18 ring carbon atoms, or an alkyl group having 1 to 15 carbon atoms, R₁-R₁₆ are each independently an aryl group having 6 to 18 ring carbon atoms, a heteroaryl group having 5 to 18 ring carbon atoms, an alkyl group having 1 to 15 carbon atoms, a silyl group, a halogen atom, a hydrogen atom or a deuterium atom, a, b, c and d are each independently an integer of 0 to 3, and L₁ is a divalent connecting group.

Ar may be an aryl group having 6 to 30 ring carbon atoms or an alkyl group having 1 to 15 carbon atoms.

At least two adjacent ones of R₁-R₁₆ may be combined to each other and form a saturated or unsaturated ring, provided that an aromatic ring is not formed by a combination of R₁ with R₅ and R₃, R₂ with R₉ and R₁₂, or R₄ with R₁₃ and R₁₆.

A dibenzofuranyl moiety or a dibenzothiophenyl moiety of Formula (3) may be bound to a phenylene moiety at position 2, position 3, or position 4 of the dibenzofuranyl moiety or the dibenzothiophenyl moiety.

The embodiments may be realized by providing an organic electroluminescence (EL) device including an anode; a cathode; and a light emitting layer between the anode and the cathode, wherein the light emitting layer includes the compound for an organic EL device according to an embodiment.

The embodiments may be realized by providing an organic electroluminescence (EL) device including an anode; a cathode; a light emitting layer between the anode and the cathode; and at least one stacking layer between the anode and the light emitting layer, wherein the at least one stacking layer includes the compound for an organic EL device according to an embodiment.

### BRIEF DESCRIPTION OF THE DRAWING

Features will be apparent to those of skill in the art by describing in detail exemplary embodiments with reference to the attached drawing in which:

FIG. 1 illustrates a schematic diagram of the structure of an organic EL device according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. Like reference numerals refer to like elements throughout.

According to an embodiment, an organic EL device driven at a low voltage and having improved hole transport properties and electron tolerance may be obtained by using a compound in which a dibenzofuranyl moiety or a dibenzothiophenyl moiety is introduced as an electron accepting substituent on a nitrogen atom of a heat resistant carbazole group having hole transport properties. In addition, a hole transport material according to an embodiment may include a compound in which two carbazole groups are introduced via a divalent connecting group, and a highest occupied molecular orbital (HOMO) level may be lowered. By using the hole transport material as a material of a light emitting layer or a stacking layer disposed between the light emitting layer and an anode, an organic EL device having long life and high efficiency may be manufactured.

The material for an organic EL device according to an embodiment may include a compound in which a dibenzofuranyl moiety or a dibenzothiophenyl moiety is introduced on a nitrogen atom of a carbazole group. In an implementation, the compound may be represented by the following Formula (1).

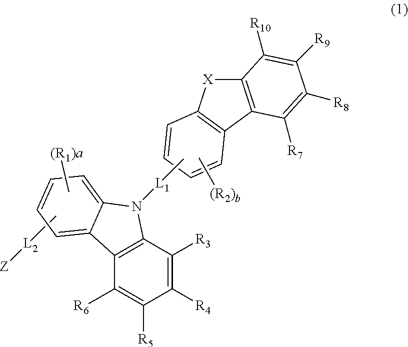

In Formula (1), X may be, e.g., an oxygen atom or a sulfur atom. Z may be or may include, e.g., an aryl group having 6 to 30 ring carbon atoms or a heteroaryl group having 5 to 30 ring carbon atoms. In an implementation, Z may include, e.g., a phenyl group, a 1-naphtyl group, a 2-naphtyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a 2-biphenylyl group, a 3-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-tolyl group, a m-tolyl group, a p-tolyl group, a p-t-butylphenyl group, a p-(2-phenylpropyl)phenyl group, a 3-methyl-2-naphtyl group, a 4-methyl-1-naphtyl group, a 4-methyl-1-anthryl group, a 4′-methylbiphenylyl group, a 4″-t-butyl-p-terphenyl-4-yl group, a fluoranthenyl group, a fluorenyl group, a 1-pyrrolyl group, a 2-pyrrolyl group, a 3-pyrrolyl group, a pyradinyl group, a 2-pyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 2-quinoxalinyl group, a 5-quinoxalinyl group, a 6-quinoxalinyl group, a 1-carbazolyl group, a 2-carbazolyl group, a 3-carbazolyl group, a 4-carbazolyl group, a 9-carbazolyl group, a 1-phenanthridinyl group, a 2-phenanthridinyl group, a 3-phenanthridinyl group, a 4-phenanthridinyl group, a 6-phenanthridinyl group, a 7-phenanthridinyl group, a 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 1,7-phenanthroline-2-yl group, a 1,7-phenanthroline-3-yl group, a 1,7-phenanthroline-4-yl group, a 1,7-phenanthroline-5-yl group, a 1,7-phenanthroline-6-yl group, a 1,7-phenanthroline-8-yl group, a 1,7-phenanthroline-9-yl group, a 1,7-phenanthroline-10-yl group, a 1,8-phenanthroline-2-yl group, a 1,8-phenanthroline-3-yl group, a 1,8-phenanthroline-4-yl group, a 1,8-phenanthroline-5-yl group, a 1,8-phenanthroline-6-yl group, a 1,8-phenanthroline-7-yl group, a 1,8-phenanthroline-9-yl group, a 1,8-phenanthroline-10-yl group, a 1,9-phenanthroline-2-yl group, a 1,9-phenanthroline-3-yl group, a 1,9-phenanthroline-4-yl group, a 1,9-phenanthroline-5-yl group, a 1,9-phenanthroline-6-yl group, a 1,9-phenanthroline-7-yl group, a 1,9-phenanthroline-8-yl group, a 1,9-phenanthroline-10-yl group, a 1,10-phenanthroline-2-yl group, a 1,10-phenanthroline-3-yl group, a 1,10-phenanthroline-4-yl group, a 1,10-phenanthroline-5-yl group, a 2,9-phenanthroline-1-yl group, a 2,9-phenanthroline-3-yl group, a 2,9-phenanthroline-4-yl group, a 2,9-phenanthroline-5-yl group, a 2,9-phenanthroline-6-yl group, a 2,9-phenanthroline-7-yl group, a 2,9-phenanthroline-8-yl group, a 2,9-phenanthroline-10-yl group, a 2,8-phenanthroline-1-yl group, a 2,8-phenanthroline-3-yl group, a 2,8-phenanthroline-4-yl group, a 2,8-phenanthroline-5-yl group, a 2,8-phenanthroline-6-yl group, a 2,8-phenanthroline-7-yl group, a 2,8-phenanthroline-9-yl group, a 2,8-phenanthroline-10-yl group, a 2,7-phenanthroline-1-yl group, a 2,7-phenanthroline-3-yl group, a 2,7-phenanthroline-4-yl group, a 2,7-phenanthroline-5-yl group, a 2,7-phenanthroline-6-yl group, a 2,7-phenanthroline-8-yl group, a 2,7-phenanthroline-9-yl group, a 2,7-phenanthroline-10-yl group, a 1-phenazinyl group, a 2-phenazinyl group, a 1-phenothiazinyl group, a 2-phenothiazinyl group, a 3-phenothiazinyl group, a 4-phenothiazinyl group, a 10-phenothiazinyl group, a 1-fenoxadinyl group, a 2-fenoxadinyl group, a 3-fenoxadinyl group, a 4-fenoxadinyl group, a 10-fenoxadinyl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 2-methylpyrrol-1-yl group, a 2-methylpyrrol-3-yl group, a 2-methylpyrrol-4-yl group, a 2-methylpyrrol-5-yl group, a 3-methylpyrrol-1-yl group, a 3-methylpyrrol-2-yl group, a 3-methylpyrrol-4-yl group, a 3-methylpyrrol-5-yl group, a 2-t-butylpyrrol-4-yl group, a 3-(2-phenylpropyl)pyrrol-1-yl group, a 2-methyl-1-indolyl group, a 4-methyl-1-indolyl group, a 2-methyl-3-indolyl group, a 4-methyl-3-indolyl group, a 2-t-butyl-1-indolyl group, a 4-t-butyl-1-indolyl group, a 2-t-butyl-3-indolyl group, a 4-t-butyl-3-indolyl group, a methyl group, an ethyl group, a propyl group, an isopropyl group, an n-butyl group, a s-butyl group, an isobutyl group, a t-butyl group, an n-pentyl group, an n-hexyl group, an n-heptyl group, an n-octyl group, a hydroxymethyl group, a 1-hydroxyethyl group, a 2-hydroxyethyl group, a 2-hydorxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-triboromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, a 2-norbornyl group, or the like.

In Formula (1), R₁-R₁₀ may each independently be or include, e.g., an aryl group having 6 to 18 ring carbon atoms, a heteroaryl group having 1 to 18 ring carbon atoms, an alkyl group having 1 to 15 carbon atoms, a silyl group, a halogen atom, a hydrogen atom or a deuterium atom. a and b may each independently be an integer of, e.g., 0 to 3.

In an implementation, the aryl group of R₁-R₁₀ may be an aryl group having 6 to 18 ring carbon atoms, e.g., a 1-naphtyl group, a 2-naphtyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a 2-biphenylyl group, a 3-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-tolyl group, a m-tolyl group, a p-tolyl group, a p-t-butylphenyl group, a p-(2-phenylpropyl)phenyl group, a 3-methyl-2-naphtyl group, a 4-methyl-1-naphtyl group, a 4-methyl-1-anthryl group, a 4′-methylbiphenylyl group, a 4″-t-butyl-p-terphenyl-4-yl group, a fluoranthenyl group, a fluorenyl group, or the like.

In an implementation, the heteroaryl group of R₁-R₁₀ may be a heteroaryl group having 5 to 18 ring carbon atoms, e.g., a 1-pyrrolyl group, a 2-pyrrolyl group, a 3-pyrrolyl group, a pyradinyl group, a 2-pyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 2-quinoxalinyl group, a 5-quinoxalinyl group, a 6-quinoxalinyl group, a 1-carbazolyl group, a 2-carbazolyl group, a 3-carbazolyl group, a 4-carbazolyl group, a 9-carbazolyl group, a 1-phenanthridinyl group, a 2-phenanthridinyl group, a 3-phenanthridinyl group, a 4-phenanthridinyl group, a 6-phenanthridinyl group, a 7-phenanthridinyl group, a 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 1,7-phenanthroline-2-yl group, a 1,7-phenanthroline-3-yl group, a 1,7-phenanthroline-4-yl group, a 1,7-phenanthroline-5-yl group, a 1,7-phenanthroline-6-yl group, a 1,7-phenanthroline-8-yl group, a 1,7-phenanthroline-9-yl group, a 1,7-phenanthroline-10-yl group, a 1,8-phenanthroline-2-yl group, a 1,8-phenanthroline-3-yl group, a 1,8-phenanthroline-4-yl group, a 1,8-phenanthroline-5-yl group, a 1,8-phenanthroline-6-yl group, a 1,8-phenanthroline-7-yl group, a 1,8-phenanthroline-9-yl group, a 1,8-phenanthroline-10-yl group, a 1,9-phenanthroline-2-yl group, a 1,9-phenanthroline-3-yl group, a 1,9-phenanthroline-4-yl group, a 1,9-phenanthroline-5-yl group, a 1,9-phenanthroline-6-yl group, a 1,9-phenanthroline-7-yl group, a 1,9-phenanthroline-8-yl group, a 1,9-phenanthroline-10-yl group, a 1,10-phenanthroline-2-yl group, a 1,10-phenanthroline-3-yl group, a 1,10-phenanthroline-4-yl group, a 1,10-phenanthroline-5-yl group, a 2,9-phenanthroline-1-yl group, a 2,9-phenanthroline-3-yl group, a 2,9-phenanthroline-4-yl group, a 2,9-phenanthroline-5-yl group, a 2,9-phenanthroline-6-yl group, a 2,9-phenanthroline-7-yl group, a 2,9-phenanthroline-8-yl group, a 2,9-phenanthroline-10-yl group, a 2,8-phenanthroline-1-yl group, a 2,8-phenanthroline-3-yl group, a 2,8-phenanthroline-4-yl group, a 2,8-phenanthroline-5-yl group, a 2,8-phenanthroline-6-yl group, a 2,8-phenanthroline-7-yl group, a 2,8-phenanthroline-9-yl group, a 2,8-phenanthroline-10-yl group, a 2,7-phenanthroline-1-yl group, a 2,7-phenanthroline-3-yl group, a 2,7-phenanthroline-4-yl group, a 2,7-phenanthroline-5-yl group, a 2,7-phenanthroline-6-yl group, a 2,7-phenanthroline-8-yl group, a 2,7-phenanthroline-9-yl group, a 2,7-phenanthroline-10-yl group, a 1-phenazinyl group, a 2-phenazinyl group, a 1-phenothiazinyl group, a 2-phenothiazinyl group, a 3-phenothiazinyl group, a 4-phenothiazinyl group, a 10-phenothiazinyl group, a 1-fenoxadinyl group, a 2-fenoxadinyl group, a 3-fenoxadinyl group, a 4-fenoxadinyl group, a 10-fenoxadinyl group, a 2-oxazolyl group, a 4-oxazolyl group, a 5-oxazolyl group, a 2-oxadiazolyl group, or the like.

In an implementation, the alkyl group of R₁-R₁₀ may be or may include, e.g., an alkyl group having 1 to 15 carbon atoms. In an implementation, R₁-R₁₅ may be the same or different, and at least two substituents may be the same.

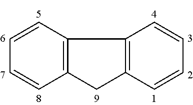 [SMILES: c1ccc2c(c1)Cc1ccccc1-2]

In an implementation, position 2, 3, or 4, e.g., position 3 or position 4, of the dibenzofuranyl moiety or the dibenzothiophenyl moiety (e.g., the moiety that includes R₂ and R₇-R₁₀ thereon) may be connected to L₁. When the nitrogen atom of the carbazole group and the oxygen atom or sulfur atom of the dibenzofuranyl moiety or the dibenzothiophenyl moiety are disposed at the para position, radical reactivity may increase. For example, at the position 4, conjugation effects corresponding to two benzene rings may be obtained, and the stability of the compound may be high, thereby realizing further long life.

In an implementation, L₁ may be a divalent connecting group or a single bond. In an implementation, L₂ may be a divalent connecting group. The divalent connecting group may include, e.g., a phenylene group or a naphthalenyl group. In an implementation, the divalent connecting group may include, e.g., a p-phenylene group or a 1,4-naphthalenyl group. In an implementation, the p-phenylene group may be used in consideration of the molecular weight of a compound. A layer of an organic EL device may be formed by depositing or coating the material or compound for an organic EL device according to an embodiment. For example, the layer may be easily formed by the deposition method by using the compound that includes the p-phenylene group having a small molecular weight.

In an implementation, when L₁ is the single bond, R₇-R₁₀ may not be a heteroaryl group, e.g., none of R₇-R₁₀ may be or include a heteroaryl group. The introduction of the heteroaryl group at R₇-R₁₀, when L₁ is the single bond, may not be desirable in consideration of the emission efficiency and life of the organic EL device.

In an implementation, a plurality of, e.g., two of, adjacent ones of R₁-R₁₀ may be combined to each other and may form a saturated or unsaturated ring. In an implementation, an aromatic ring may not be formed via the combination of R₁ with R₃ and/or R₆, or R₂ with R₇ and/or R₁₀.

The compound for an organic EL device may exhibit hole transport properties, and the hole transport properties and electron tolerance may be improved by introducing the electron accepting substituent (e.g., the dibenzofuranyl group or moiety or the dibenzothiophenyl group or moiety) on the nitrogen atom of the strongly heat resistant carbazole group.

In an implementation, the compound for an organic EL device may include a dibenzofuranyl moiety or a dibenzothiophenyl moiety on the nitrogen atom of a carbazole group. In an implementation, the compound according to an embodiment may be represented by the following Formula (2).

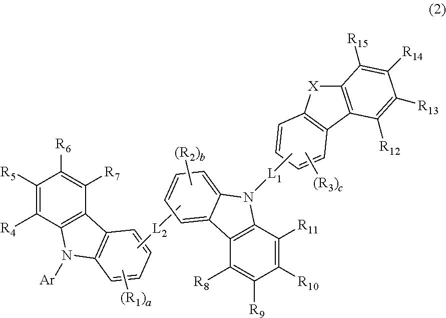

In Formula (2), X may be an oxygen atom or a sulfur atom. In an implementation, Ar may be or may include, e.g., an aryl group having 6 to 18 ring carbon atoms, a heteroaryl group having 5 to 18 ring carbon atoms, or an alkyl group having 1 to 15 carbon atoms. In an implementation, Ar may include, e.g., an aryl group having 6 to 18 ring carbon atoms or an alkyl group having 1 to 15 carbon atoms.

R₁-R₁₅ may each independently be or include, e.g., an aryl group having 6 to 18 ring carbon atoms, an alkyl group having 1 to 15 carbon atoms, a silyl group, a halogen atom, a hydrogen atom, or a deuterium atom. a, b, and c may each independently be an integer of, e.g., 0 to 3.

In an implementation, R₁-R₁₅ may each independently be or include, e.g., an aryl group having 6 to 18 ring carbon atoms or an alkyl group having 1 to 15 carbon atoms. R₁-R₁₅ may be different from each other, and at least two thereof may be the same substituents.

As described above, in an implementation, the position 3 or position 4 of the dibenzofuranyl moiety or the dibenzothiophenyl moiety (e.g., the moiety that includes R₃ and R₁₂ to R₁₅) may be connected with L₁.

In an implementation, Ar in the above Formula (2) may include a group represented by one of the following Formulae c-1 to c-16.

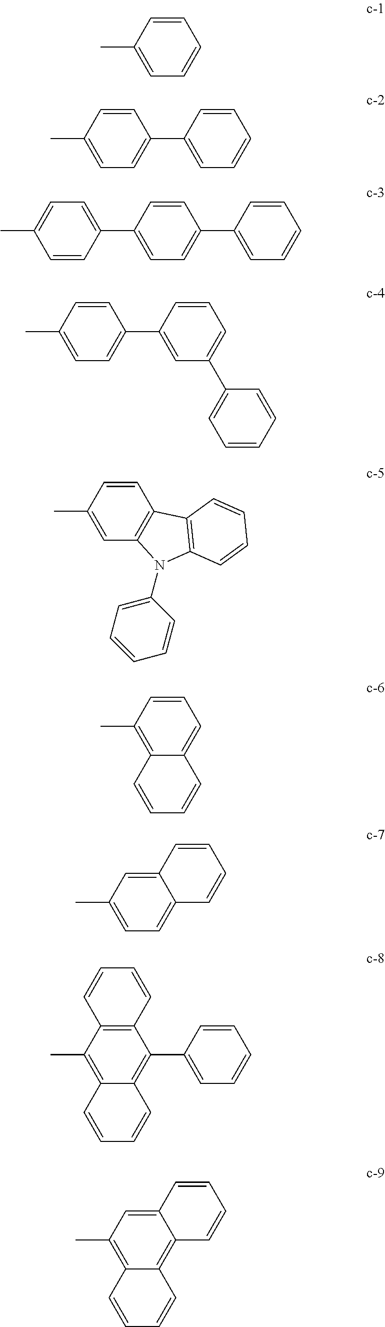

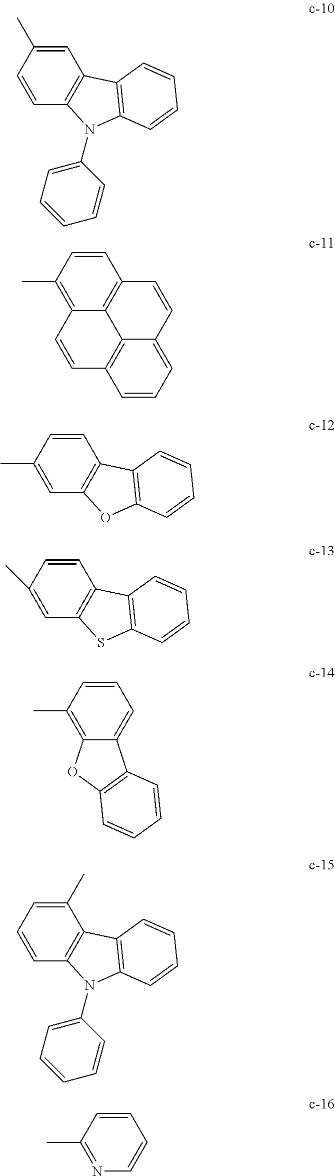

In an implementation, Ar in the above Formula (2) may include a group represented by one of the following Formulae c-17 to c-25.

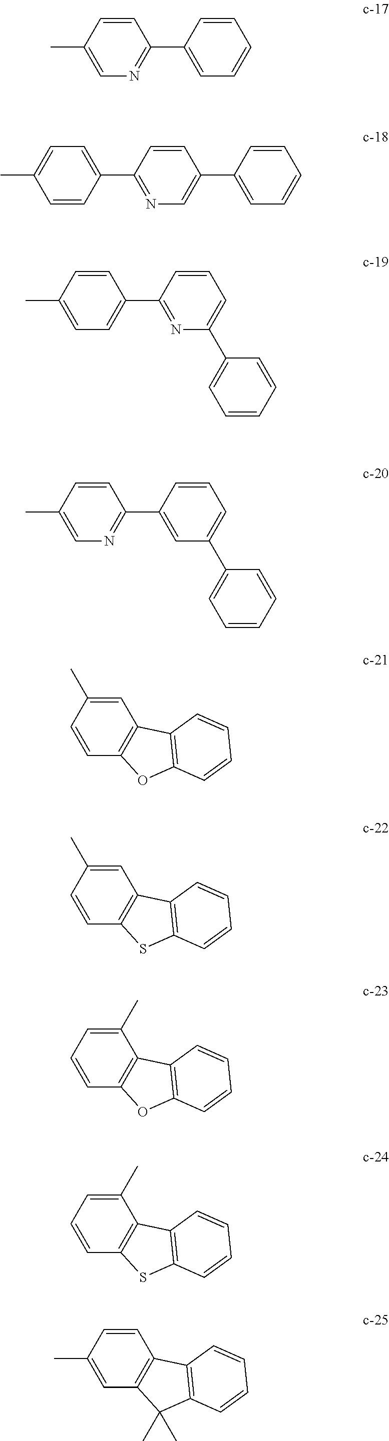

In an implementation, L₁ may be a divalent connecting group or a single bond, and L₂ may be a divalent connecting group. In an implementation, the divalent connecting group may include, e.g., a phenylene group or a naphthalenyl group are preferable. In an implementation, the divalent connecting group may include, e.g., a p-phenylene group or a 1,4-naphthalenyl group. In an implementation, the p-phenylene group may be used in consideration of a molecular weight. A layer of an organic EL device may be formed by depositing or coating the material or compound for an organic EL device according to an embodiment. For example, the p-phenylene group having a small molecular weight may be appropriately used in a deposition method.

In an implementation, a plurality of, e.g., two of, adjacent ones of R₁-R₁₅ may be combined to each other and form a saturated or unsaturated ring. In an implementation, an aromatic ring may not be formed via the combination of R₁ with R₄ and/or R₇, R₂ with R₈ and/or R₁₁, or R₃ with R₁₂ and/or R₁₅.

In the compound for an organic EL device according to an embodiment, two strongly heat resistant carbazole groups may be included, and hole transport properties and electron tolerance may be improved by introducing the electron accepting substituents (e.g., the dibenzofuranyl moiety or the dibenzothiophenyl moiety) on the nitrogen atom of one carbazole group.

In an implementation, the compound for an organic EL device according to an embodiment may include a compound that has a dibenzofuranyl moiety or a dibenzothiophenyl moiety on the nitrogen atom of a carbazole group. In an implementation, the compound for an organic EL device may represented by the following Formula (3).

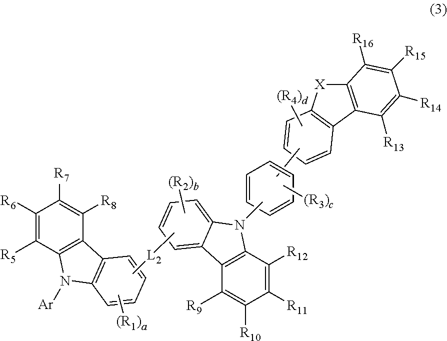

In Formula (3), X may be, e.g., an oxygen atom or a sulfur atom. Ar may be or may include, e.g., an aryl group having 6 to 18 ring carbon atoms, a heteroaryl group having 1 to 30 ring carbon atoms, or an alkyl group having 1 to 15 carbon atoms. In an implementation, Ar may be, e.g., an aryl group having 6 to 18 ring carbon atoms or an alkyl group having 1 to 15 carbon atoms. In an implementation, Ar may include, e.g., the aryl group having 6 to 18 ring carbon atoms, the heteroaryl group having 5 to 18 ring carbon atoms, or an alkyl group having 1 to 15 carbon atoms.

R₁-R₁₆ may each independently be or include, e.g., an aryl group having 6 to 18 ring carbon atoms, a heteroaryl group having 5 to 18 ring carbon atoms, an alkyl group having 1 to 15 carbon atoms, a halogen atom, a hydrogen atom, or a deuterium atom. a, b, c, and d may each independently be, e.g., an integer of 0 to 3.

In an implementation, R₁-R₁₉ may be or may include, e.g., an aryl group having 6 to 30 ring carbon atoms, a heteroaryl group having 5 to 30 ring carbon atoms, or an alkyl group having 1 to 15 carbon atoms. In an implementation, R₁-R₁₉ may be different from each other, and at least two thereof may be the same substituents.

In an implementation, Ar in the above Formula (3) may include a group represented by one of the following Formulae c-1 to c-16.

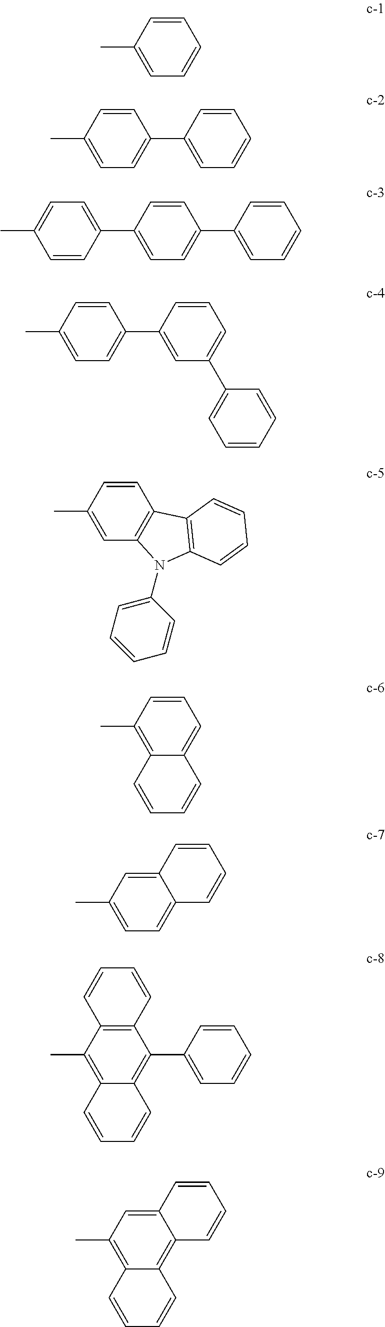

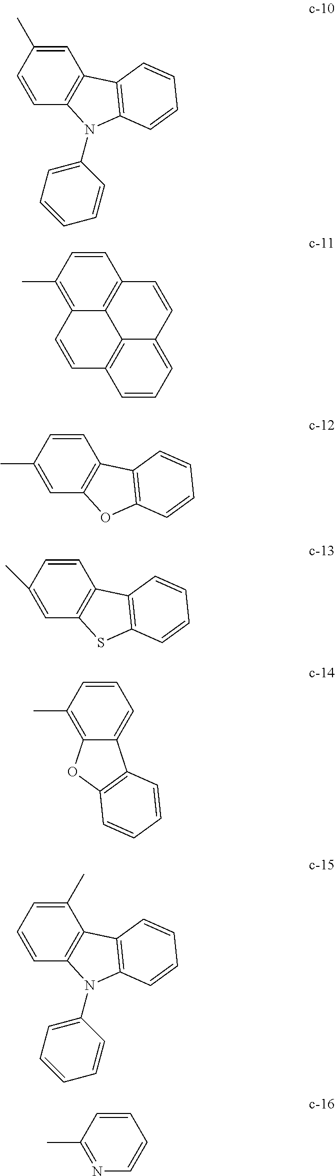

In an implementation, Ar in the above Formula (3) may include a group represented by one of the following Formulae c-17 to c-25.

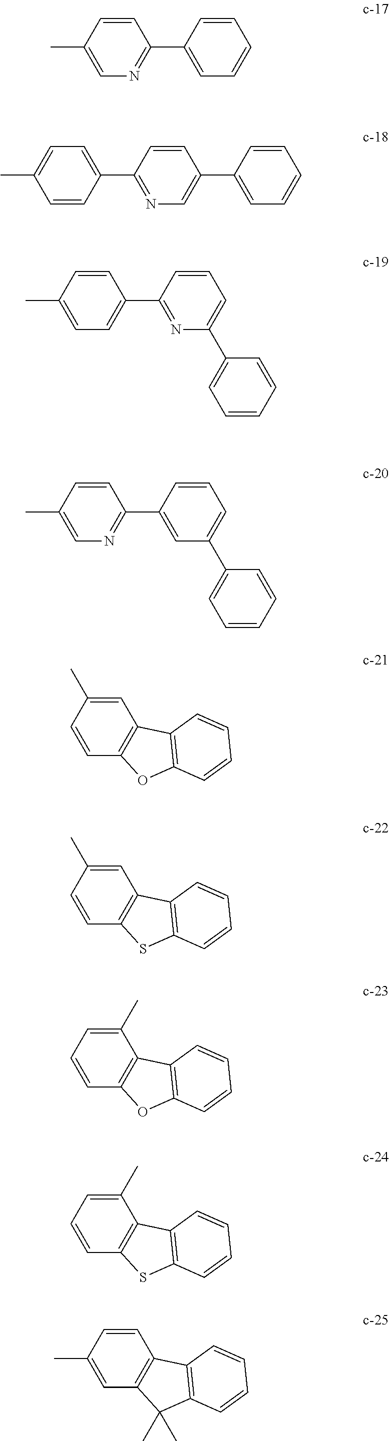

As described above, in an implementation, the position 3 or position 4 of the dibenzofuranyl moiety or the dibenzothiophenyl moiety (e.g., the moiety that includes R₄ and R₁₃ to R₁₆) may be connected with L₁.

In an implementation, L₁ may be a divalent connecting group. In an implementation, the divalent connecting group may include, e.g., a phenylene group or a naphthalenyl group. In an implementation, the divalent connecting group may include, e.g., a p-phenylene group or a 1,4-naphthalenyl group. In an implementation, the p-phenylene group may be used in consideration of a molecular weight. A layer of an organic EL device may be formed by depositing or coating the material or compound for an organic EL device. In this case, the p-phenylene group (having a small molecular weight) may be appropriately used for in a deposition method.

In an implementation, a plurality (e.g., two) of adjacent ones of R₁-R₁₆ may be combined to each other and form a saturated or unsaturated ring. In an implementation, an aromatic ring may not be formed via the combination of R₁ with R₅ and/or R₈, R₂ with R₉ and/or R₁₂, or R₄ with R₁₃ and/or R₁₆.

The compound for an organic EL device according to an embodiment may exhibit hole transport properties, may include two strongly heat resistant carbazole groups, may introduce the electron accepting substituents of the dibenzofuranyl moiety or the dibenzothiophenyl moiety on the nitrogen atom of one carbazole group, and hole transport properties and electron tolerance may be improved. In an implementation, the nitrogen atom of the carbazole group may be with the dibenzofuranyl moiety or the dibenzothiophenyl moiety via a phenylene group, and radical reactivity (due to the nitrogen atom with the oxygen atom or the sulfur atom) may be restrained through the conjugation of a benzene ring, thereby realizing long life. For example, marked effects may be obtained in a blue emitting region.

The compound for an organic EL device according to an embodiment may include one of the following Compounds 1 to 50.

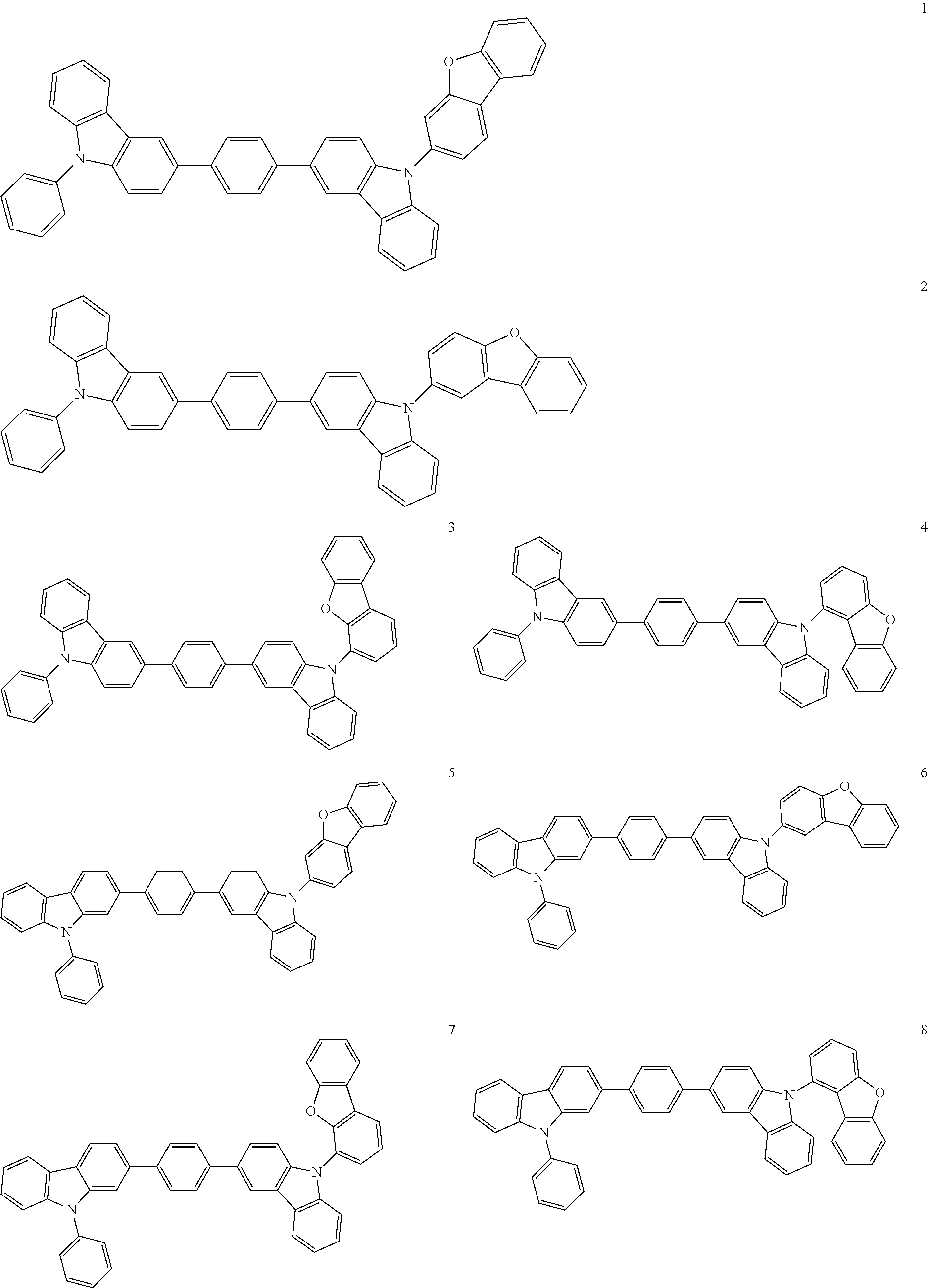

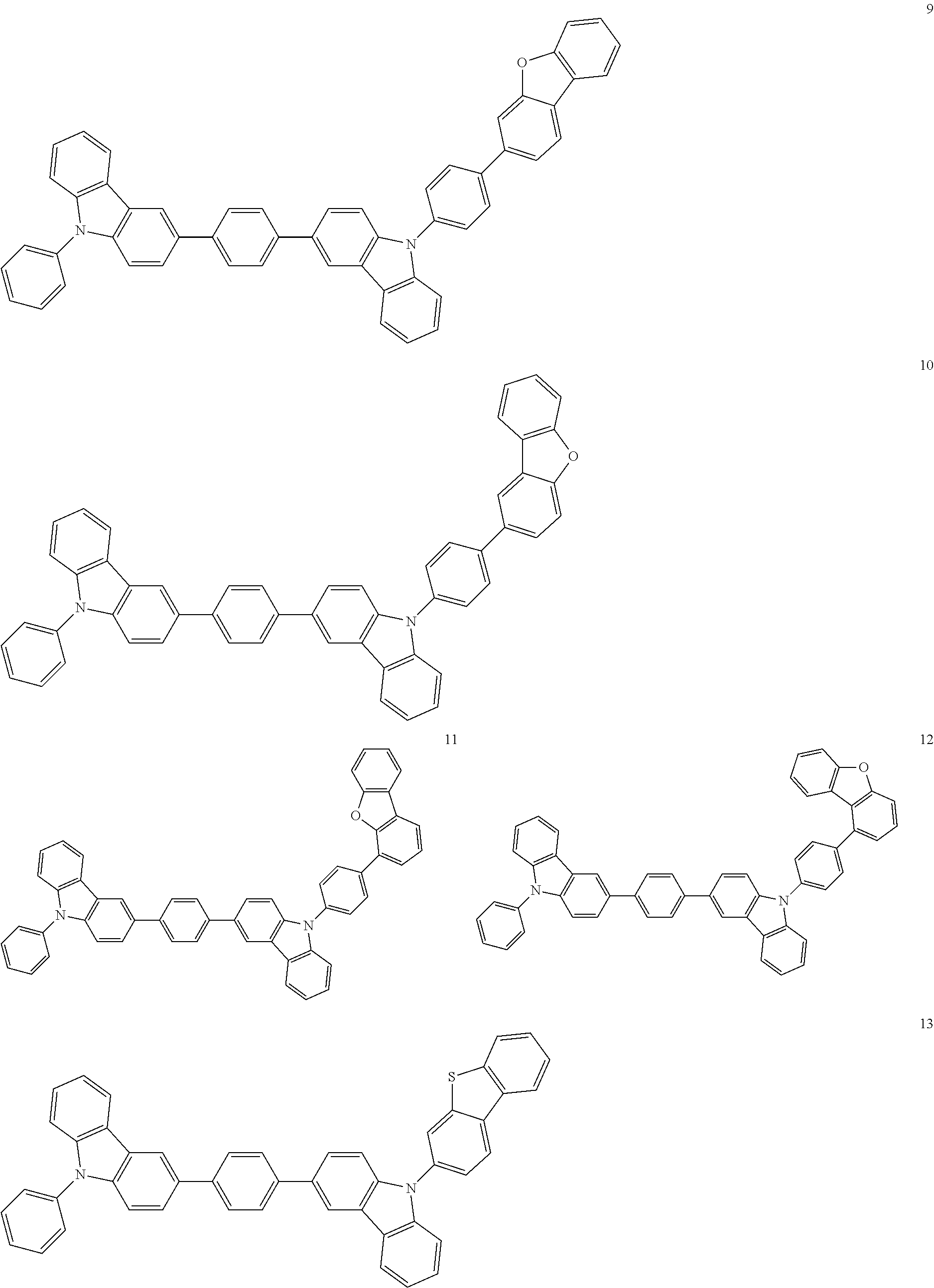

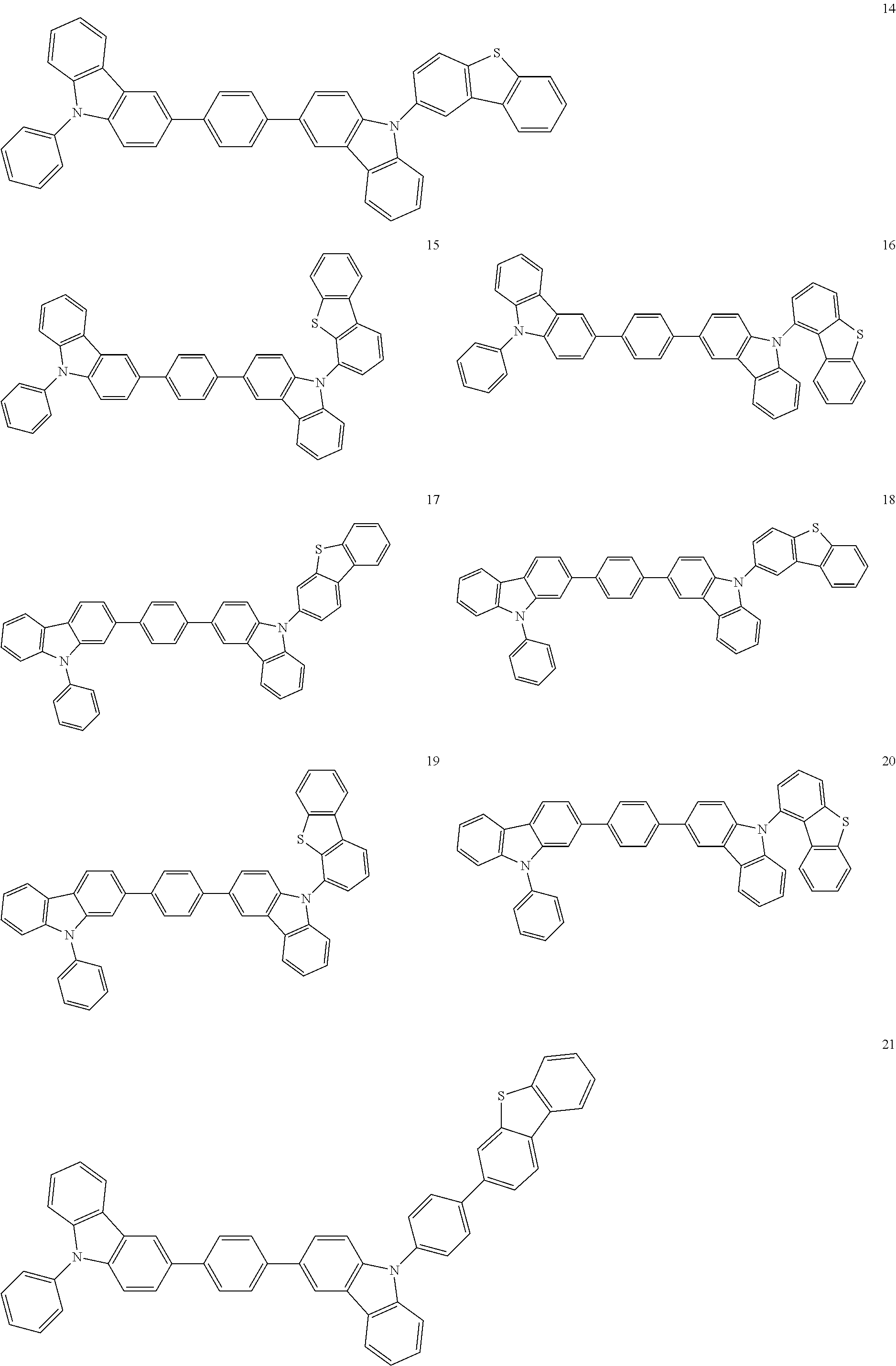

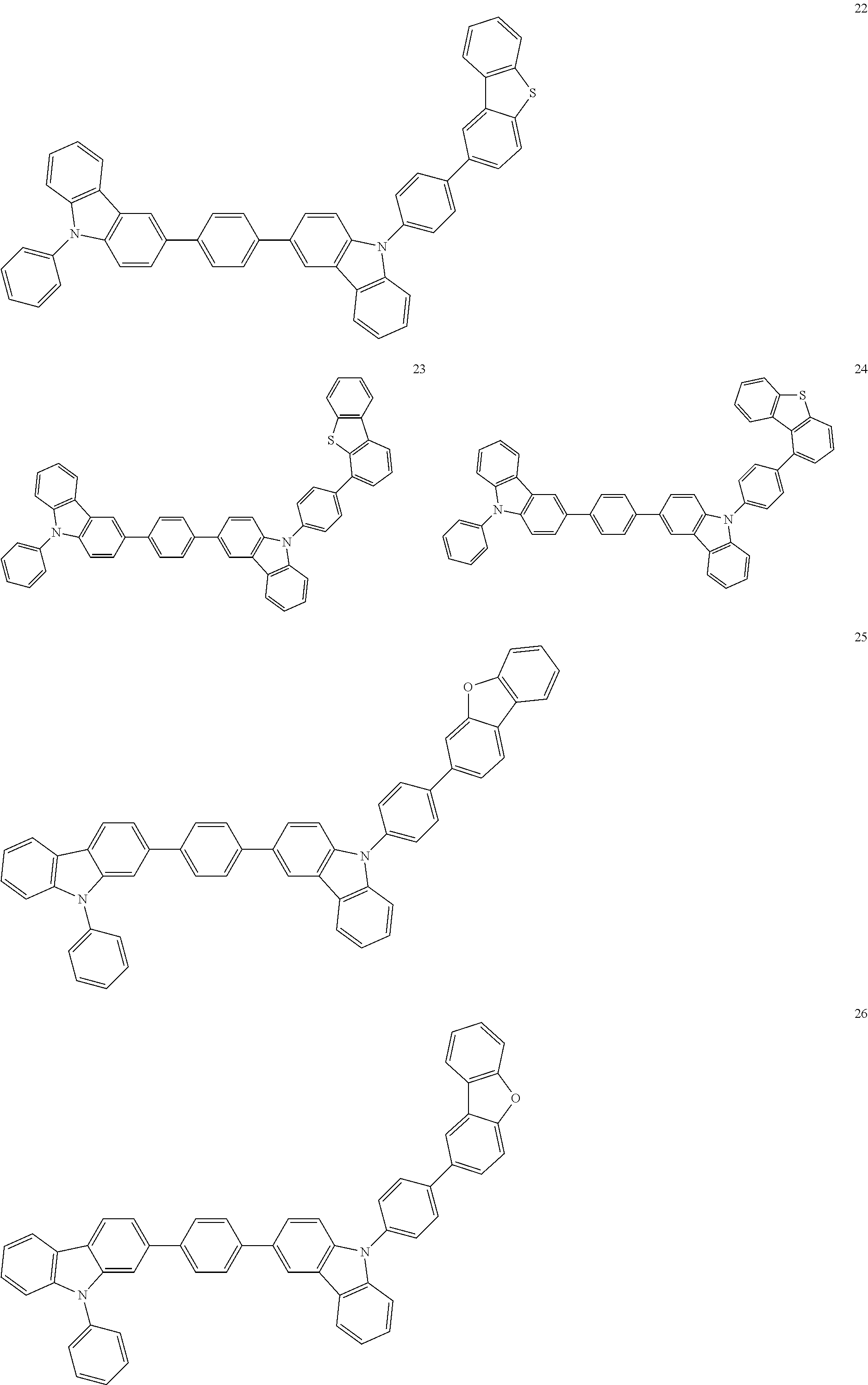

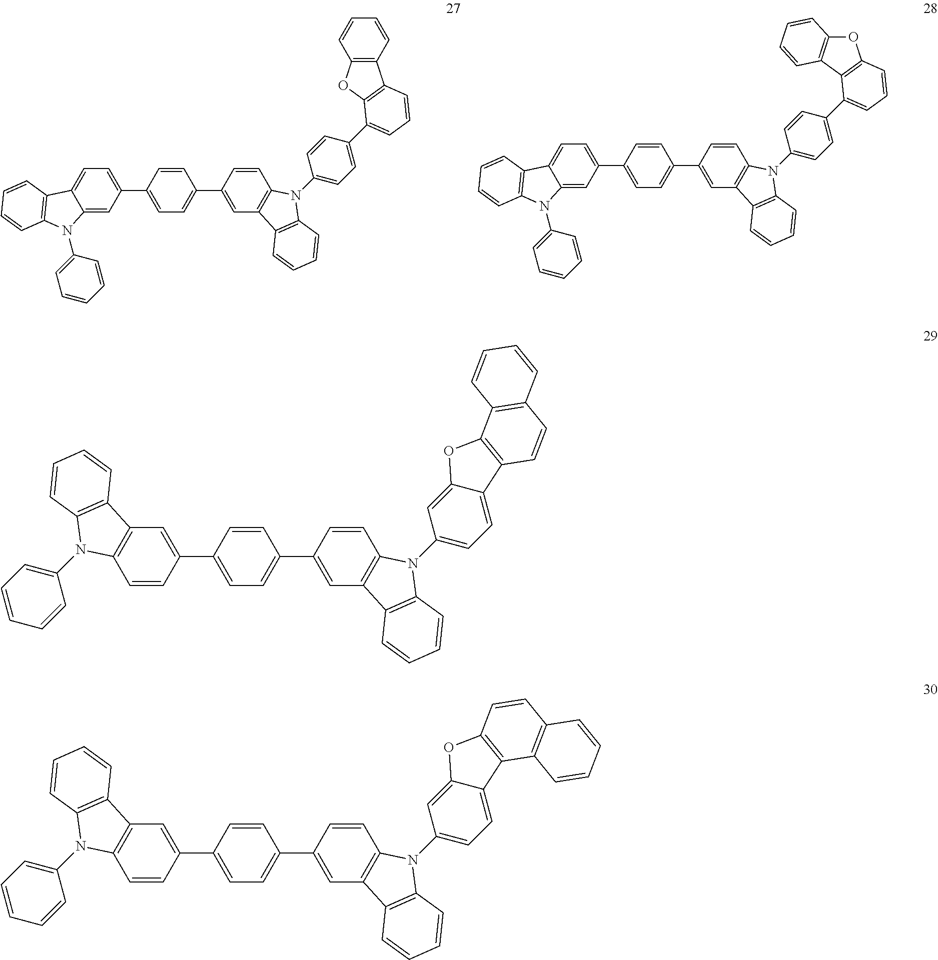 [SMILES: c1ccc(-n2c3ccccc3c3ccc(-c4ccc(-c5ccc6c(c5)c5ccccc5n6-c5ccc(-c6cccc7c6oc6ccccc67)cc5)cc4)cc32)cc1 ; c1ccc(-n2c3ccccc3c3ccc(-c4ccc(-c5ccc6c(c5)c5ccccc5n6-c5ccc(-c6cccc7oc8ccccc8c67)cc5)cc4)cc32)cc1 ; c1ccc(-n2c3ccccc3c3cc(-c4ccc(-c5ccc6c(c5)c5ccccc5n6-c5ccc6c(c5)oc5c7ccccc7ccc65)cc4)ccc32)cc1 ; c1ccc(-n2c3ccccc3c3cc(-c4ccc(-c5ccc6c(c5)c5ccccc5n6-c5ccc6c(c5)oc5ccc7ccccc7c56)cc4)ccc32)cc1]

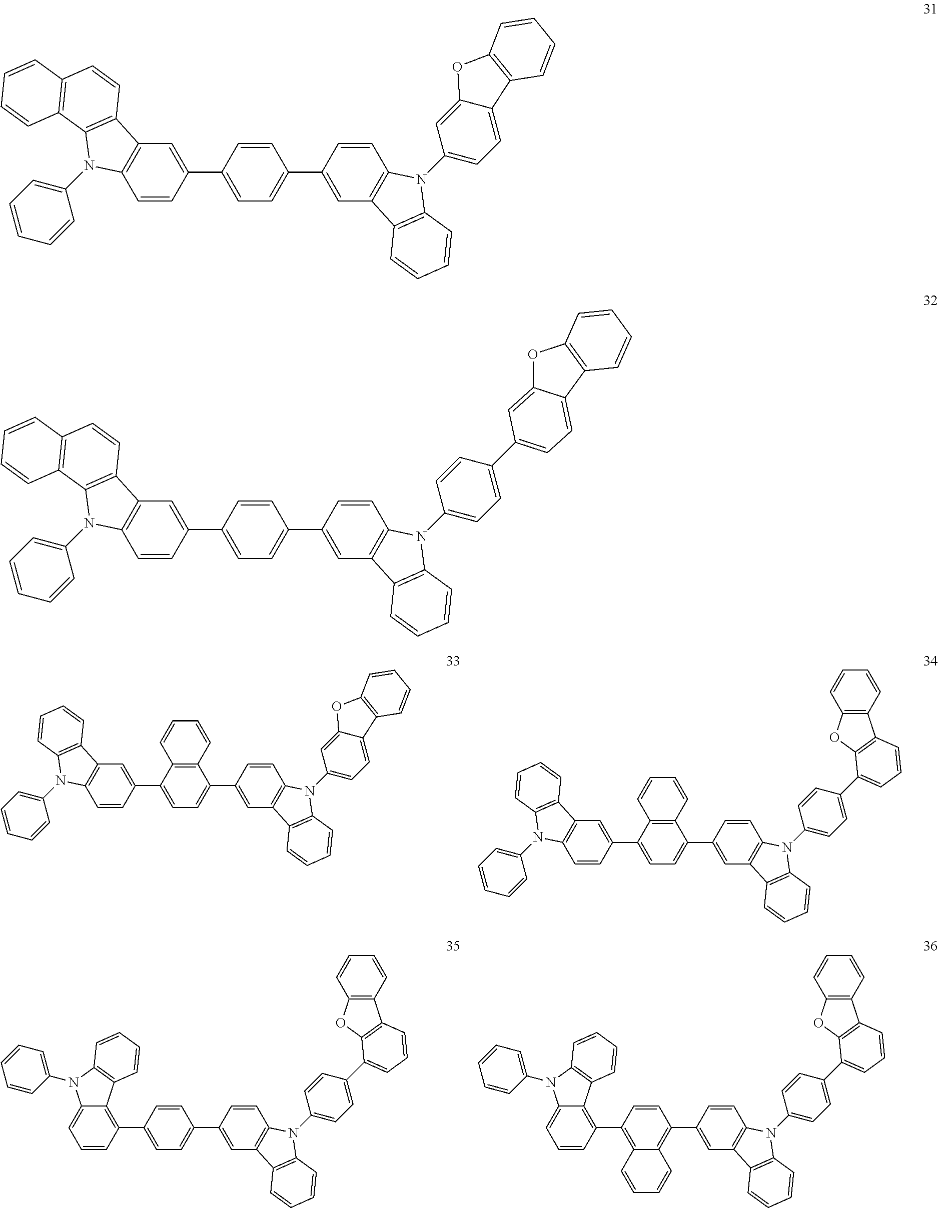

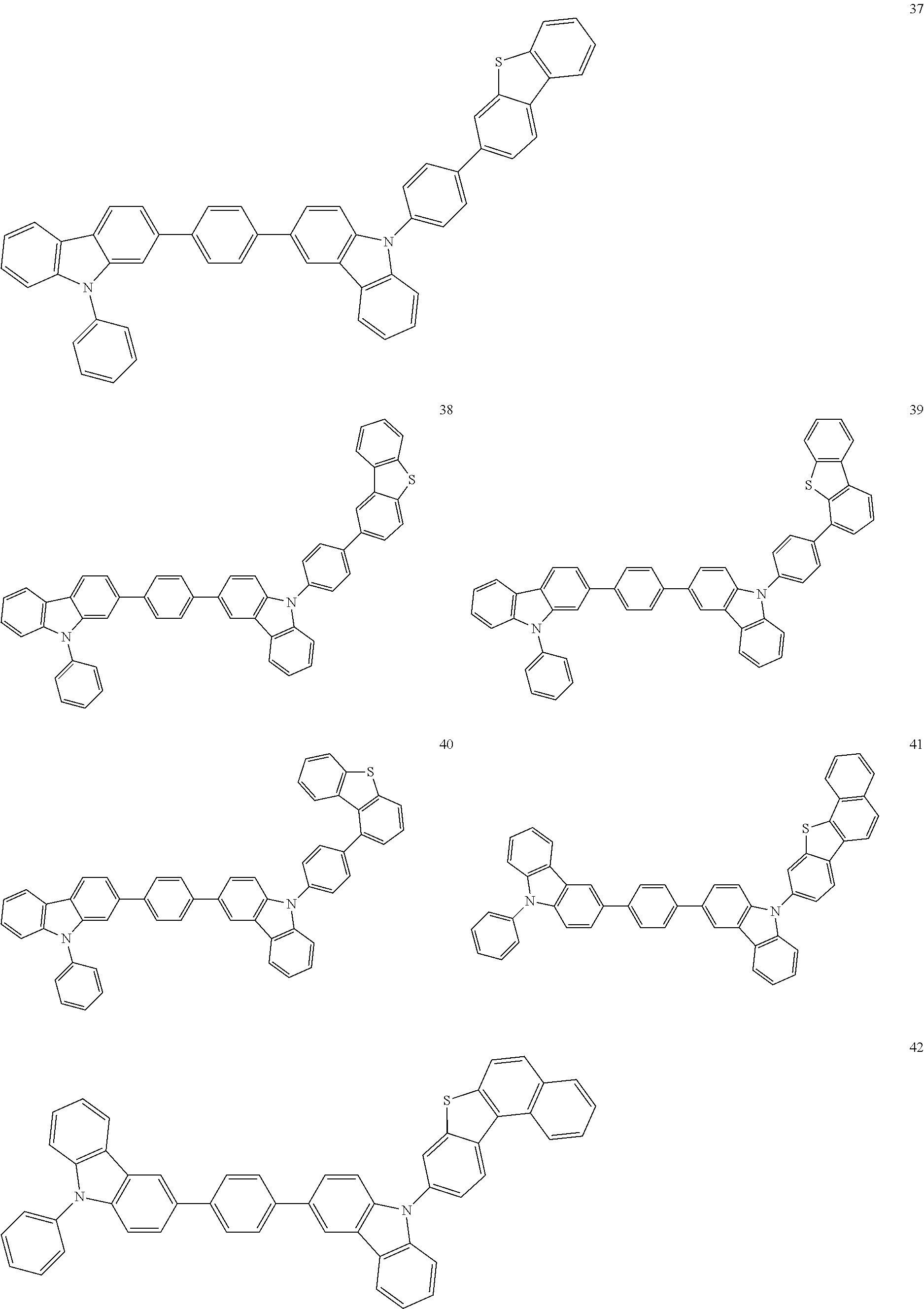

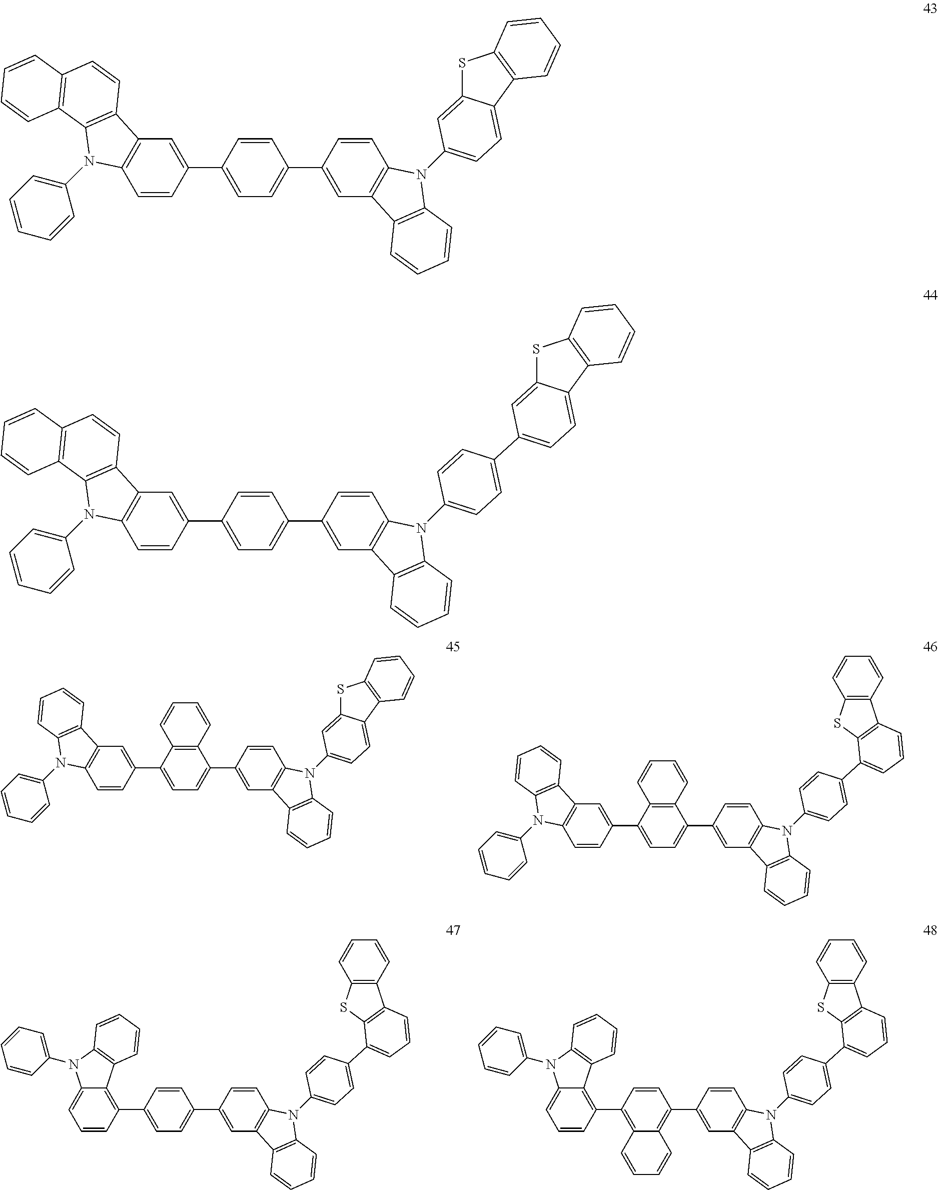

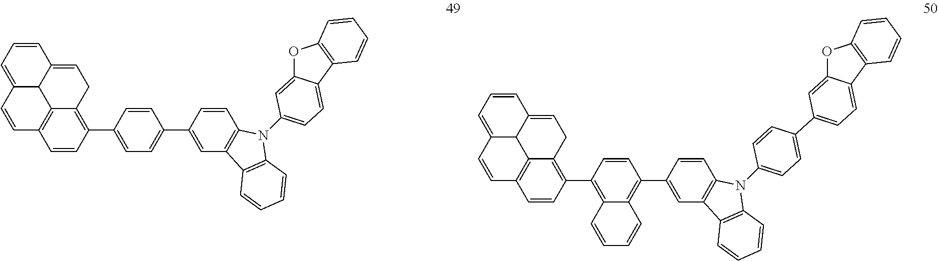 [SMILES: C1=CC2=CCc3c(-c4ccc(-c5ccc6c(c5)c5ccccc5n6-c5ccc6c(c5)oc5ccccc56)cc4)ccc4c3C2C(=C1)C=C4 ; C1=CC2=CCc3c(-c4ccc(-c5ccc6c(c5)c5ccccc5n6-c5ccc(-c6ccc7c(c6)oc6ccccc67)cc5)c5ccccc45)ccc4c3C2C(=C1)C=C4]

In an implementation, the compound for an organic EL device may be appropriately used in a light emitting layer of an organic device. In an implementation, the compound for an organic EL device may be used in one layer among stacking layers disposed between a light emitting layer and an anode. Thus, hole transport properties may be improved, and the driving of the organic EL device at a low voltage with high efficiency may be realized.

(Organic EL Device)

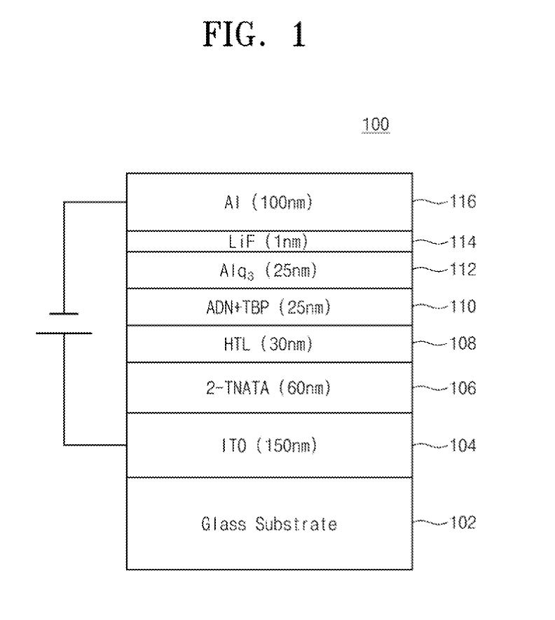

An organic EL device using the material or compound for an organic EL device according to an embodiment will be explained. FIG. 1 illustrates a schematic diagram of the configuration of an organic EL device 100 according to an embodiment. The organic EL device 100 may include, e.g., a substrate 102, an anode 104, a hole injection layer 106, a hole transport layer 108, a light emitting layer 110, an electron transport layer 112, an electron injection layer 114, and a cathode 116. In an implementation, the compound for an organic EL device may be used in a light emitting layer of an organic EL device. n an implementation, the compound for an organic EL device may be used in a layer of the stacking layers between a light emitting layer and an anode.

For example, an example using the compound for an organic EL device in the hole transport layer 108 will be explained. The substrate 102 may be, e.g., a substrate formed by using glass, silicon, a resin, etc., or a flexible substrate. The anode 104 may be disposed on the substrate 102 and may be formed by using indium tin oxide (ITO), indium zinc oxide (IZO), etc. The hole injection layer 106 may be disposed on the anode 104 and may include, for example, 4,4′,4″-tris[2-naphtyl(phenyl)amino]-triphenylamine (2-TNATA) or [N,N,N′,N′-tetrakis(3-methylphenyl)-3,3′-dimethylbenzidine) (HMTPD), etc. The hole transport layer 108 may be disposed on the hole injection layer 106 and may be formed by using the compound for an organic EL device according to an embodiment. In an implementation, the light emitting layer 110 may be disposed on the hole transport layer 108 and may be formed by using the compound for an organic EL device. In an implementation, the light emitting layer 110 may be formed by doping 2,5,8,11-tetra-t-butylperylene (TBP) in a host material including, e.g., 9,10-di(2-naphtyl)anthracene (ADN). The electron transport layer 112 may be disposed on the light emitting layer 110 and may be formed by using, e.g., tris(8-hydroxyquinolinato)aluminum (Alq₃). The electron injection layer 114 may be disposed on the electron transport layer 112 and may be formed using a material including, e.g., lithium fluoride (LiF). The cathode 116 may be disposed on the electron injection layer 114 and may be formed using a metal such as Al or a transparent material such as ITO, IZO, etc. The above-described thin layers may be formed by selecting an appropriate layer forming method such as vacuum deposition, sputtering, various coatings, etc.

In the organic EL device 100 according to this embodiment, a hole transport layer realizing driving at a low voltage and high efficiency may be formed by using the compound for an organic EL device. In an implementation, the compound for an organic EL device may be applied in an organic EL apparatus of an active matrix using thin film transistors (TFT).

In an implementation, in the organic EL device 100 according to this embodiment, the driving at a low voltage with high efficiency and long life may be realized by using the compound for an organic EL device in a light emitting layer or a layer among stacking layers between the light emitting layer and an anode.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Examples

### Preparation Method

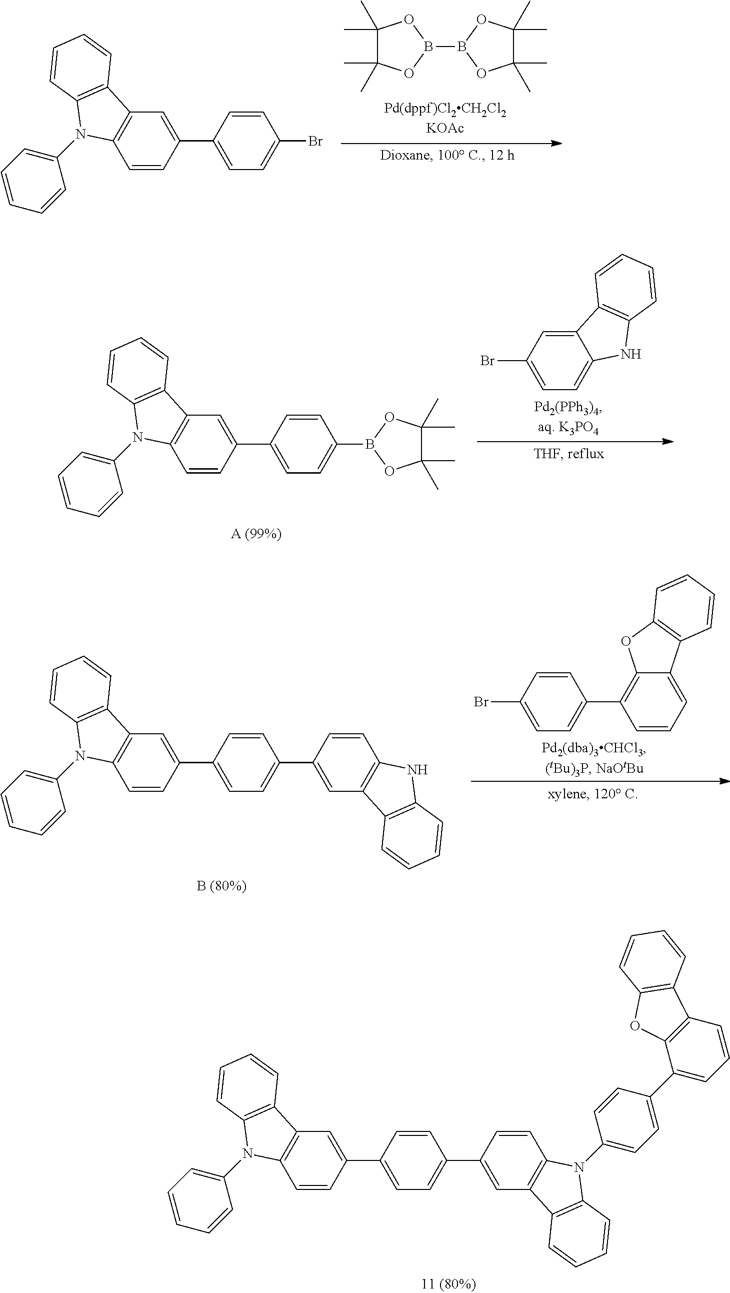

### Synthesis of Compound A

Under an argon atmosphere, 45.0 g of 3-(4-bromophenyl)-9-phenyl-9H-carbazole, 6.46 g of Pd(dppf)Cl₂.CH₂Cl₂, 33.3 g of KOAc, and 33.0 g of bis(pinacolato)diboron were added to 750 mL of a dioxane solvent in a 2 L flask, and air was removed to attain a vacuum state, followed by stirring at 100° C. for 12 hours. Then, the solvent was removed by distillation, CH₂Cl₂ and water were added, and an organic phase was separated. Magnesium sulfate and activated carbon were added thereto, the resultant mixture was filtered with suction, and solvent was distilled off. The crude product thus obtained was separated by silica gel column chromatography (CH₂Cl₂/hexane=1/3) to produce 49.1 g of Compound A as a white solid (yield 99%).

(Identification of Compound A)

The identification of Compound A was conducted by measuring ¹H-NMR and FAB-MS. In addition, the identification of the following Compound B, Compound C, and Compound 3 was also conducted by measuring ¹H-NMR and FAB-MS as Compound A. The identification of Compound D was conducted by measuring ¹H-NMR and LC-MS. In addition, for the measurement of ¹H-NMR, a CDCl₃ solvent was used.

The chemical shift values of Compound A measured by ¹H-NMR were 8.47 (d, 1H), 8.40 (d, 2H), 8.23 (d, 1H), 7.89 (d, 1H), 7.75-7.79 (m, 1H), 7.59-7.64 (m, 4H), 7.42-7.52 (m, 4H), 7.25-7.36 (m, 1H), 1.58 (s, 2H).

### Synthesis of Compound B

Under an argon atmosphere, 53.9 g of pinacol borate, 38.7 g of 3-bromocarbazole, 9.79 g of Pd(PPh₃)₄, 51.4 g of K₃PO₄ and 108 mL of H₂O were added to 485 mL of a THF solvent in a 2 L flask, and air was removed to attain a vacuum state, followed by refluxing for 8 hours. After cooling in air, an organic layer was separated, and magnesium sulfate and activated clay were added. The resultant mixture was filtered with suction, and the solvent was removed by distillation. The crude product thus obtained was washed with hot hexane, CH₂Cl₂, and AcOEt to obtain 46.9 g of Compound B as a yellow solid (yield 80%).

(Identification of Compound B)

The molecular weight of Compound B measured by FAB-MS was 484.

### Synthesis of Compound 11

Under an argon atmosphere, 9.70 g of Compound B, 7.11 g of 4-(4-bromobiphenyl)dibenzofuran, 1.45 g of tris(dibenzylideneacetone)dipalladium(O) (Pd₂(dba)₃)), 510 mg of tri-tert-butyl phosphine ((t-Bu)₃P), 5.77 g of sodium-tert-butoxide were added to a 50 mL xylene solvent in a 500 mL three-necked flask, followed by heating and stirring at 120° C. for 12 hours. After cooling the reactant in air, water was added, and an organic layer was separated. Then, the solvent was distilled off. The crude product thus obtained was separated by silica gel column chromatography (using a mixed solvent of dichloromethane and hexane), and recrystallized using a mixed solvent of toluene/hexane to obtain 11.6 g of Compound 11 as a white solid (yield 80%).

(Identification of Compound 11)

The chemical shift values of Compound 11 measured by ¹H-NMR were 8.86-8.89 (m, 211), 8.65-8.70 (m, 3H), 8.51 (d, 1H), 8.43 (q, 2H), 8.20-8.30 (m, 2H), 7.80-7.88 (m, 5H), 7.51-7.76 (m, 12H), 7.20-7.49 (m, 7H). In addition, the molecular weight of Compound 11 measured by FAB-MS was 727.

Organic EL devices of Examples 1 to 14 were manufactured by using the following Compounds 1-3, 9-11, 13-15, 21-23, 34, and 46 as hole transporting materials, according to the above-described manufacturing methods.

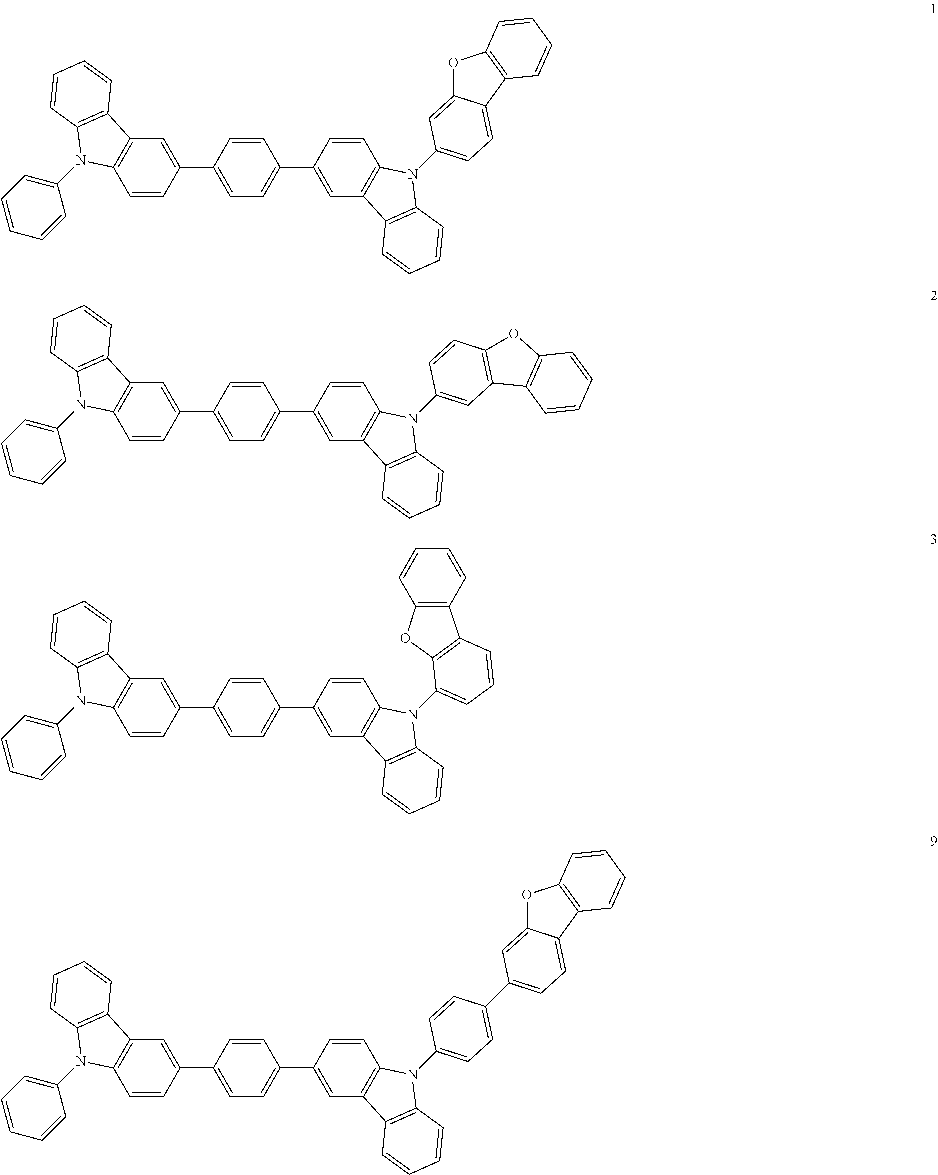

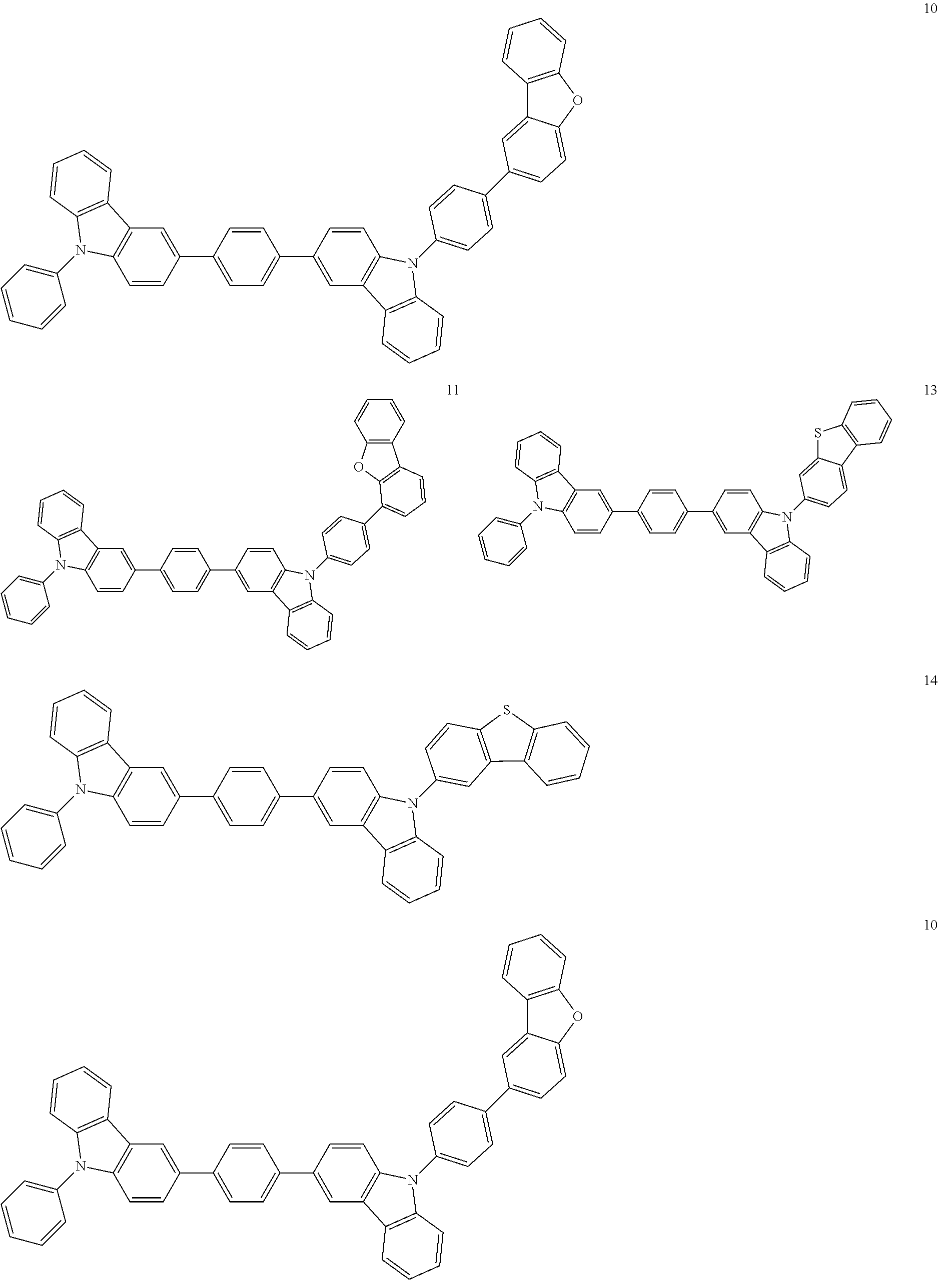

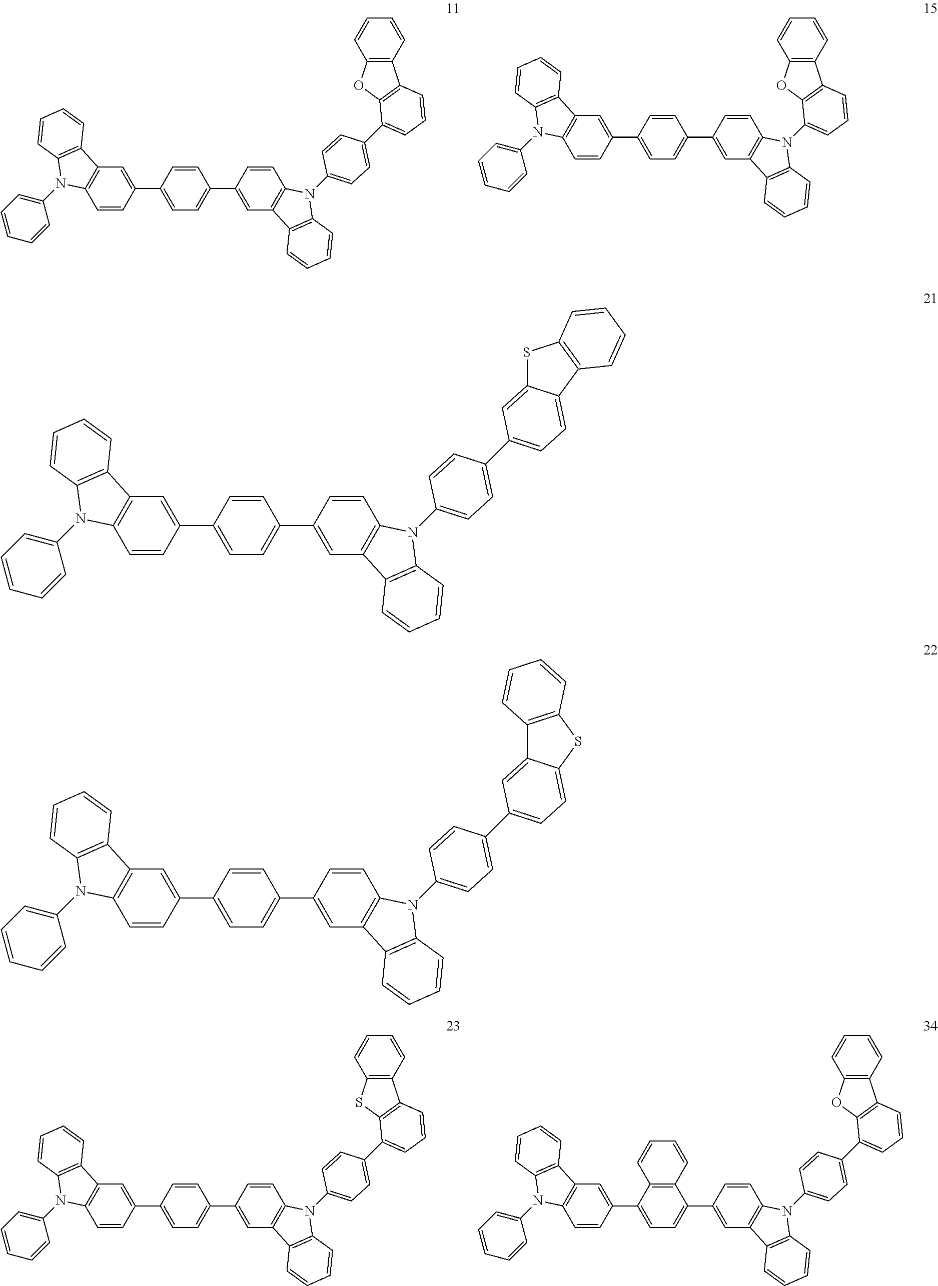

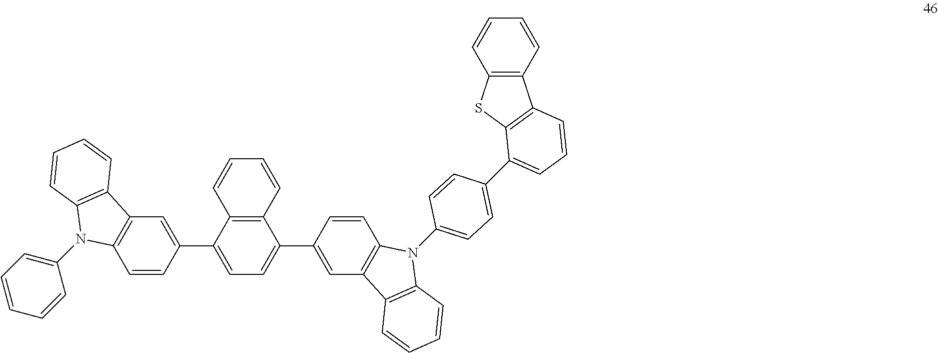 [SMILES: c1ccc(-n2c3ccccc3c3cc(-c4ccc(-c5ccc6c(c5)c5ccccc5n6-c5ccc(-c6cccc7c6sc6ccccc67)cc5)c5ccccc45)ccc32)cc1]

In addition, organic EL devices of Comparative Examples 1 to 6 were manufactured using the following Compounds 52-57 as hole transporting materials.

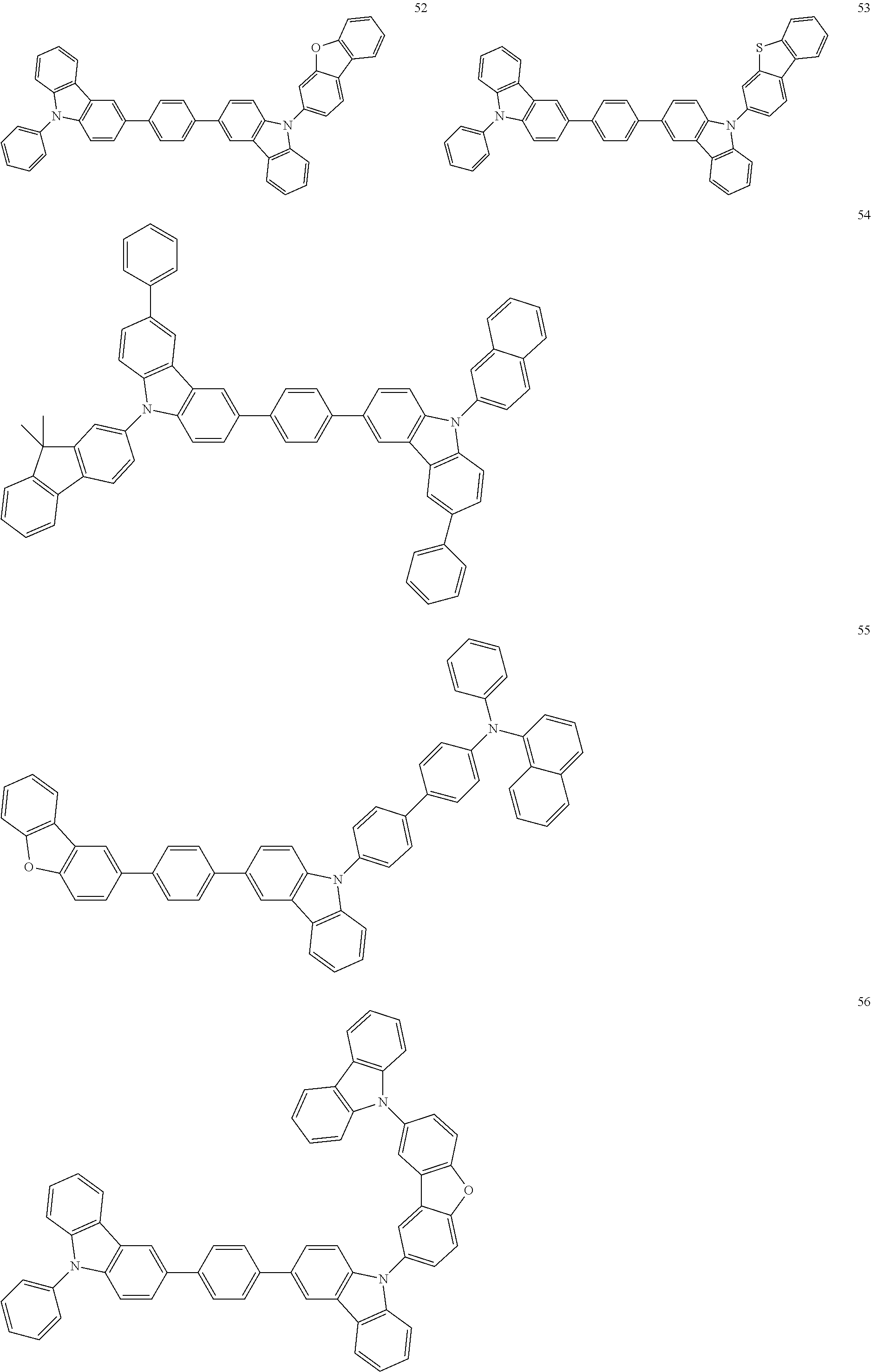

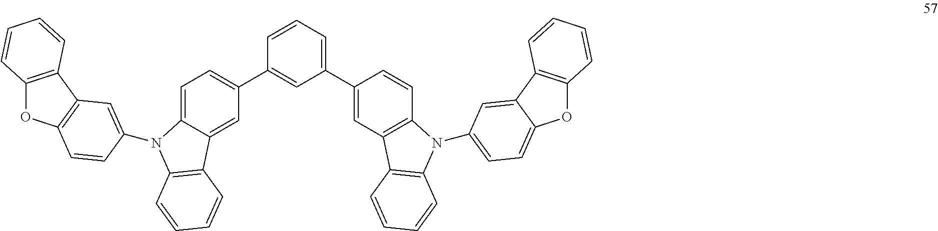 [SMILES: c1cc(-c2ccc3c(c2)c2ccccc2n3-c2ccc3oc4ccccc4c3c2)cc(-c2ccc3c(c2)c2ccccc2n3-c2ccc3oc4ccccc4c3c2)c1]

The substrate 102 was formed by using a transparent glass substrate, the anode 104 was formed using ITO to a thickness of about 150 nm, the hole injection layer 106 was formed using 2-TNATA to a thickness of about 60 nm, the hole transport layer 108 was formed using the compounds of the Examples and the Comparative Examples to a thickness of about 30 nm, the light emitting layer 110 was formed using a material obtained by doping about 3% of TBP in ADN to a thickness of about 25 nm, the electron transport layer 112 was formed using Alq₃ to a thickness of about 25 nm, the electron injection layer 114 was formed using LiF to a thickness of about 1 nm, and the cathode 116 was formed using Al to a thickness of about 100 nm.

With respect to the organic EL devices thus manufactured, voltage, emission efficiency, and life were evaluated. In addition, the evaluation was performed with current density of 10 mA/cm².

TABLE 1

| Examples of | | | | | |
|---|---|---|---|---|---|
| manufac- | | Current | Volt- | Emission | |
| turing | Hole transport | density | age | efficiency | Life |
| devices | materials | (mA/cm²) | (V) | (cd/A) | (LT50 (h)) |
| Example 1 | Compound 1 | 10 | 5.7 | 8.3 | 4,000 |
| Example 2 | Compound 2 | 10 | 5.9 | 8.9 | 1,800 |
| Example 3 | Compound 3 | 10 | 6.1 | 7.1 | 2,900 |
| Example 4 | Compound 9 | 10 | 4.7 | 6.4 | 4,300 |
| Example 5 | Compound 10 | 10 | 4.8 | 8.9 | 3,300 |
| Example 6 | Compound 11 | 10 | 5.1 | 8.1 | 4,100 |
| Example 7 | Compound 13 | 10 | 5.9 | 8.0 | 3,800 |
| Example 8 | Compound 14 | 10 | 5.9 | 8.2 | 2,300 |
| Example 9 | Compound 15 | 10 | 6.3 | 7.0 | 2,500 |
| Example 10 | Compound 21 | 10 | 4.9 | 8.5 | 4,100 |
| Example 11 | Compound 22 | 10 | 4.9 | 9.1 | 3,100 |
| Example 12 | Compound 23 | 10 | 5.3 | 8.3 | 4,000 |
| Example 13 | Compound 34 | 10 | 5.5 | 8.1 | 4,000 |
| Example 14 | Compound 46 | 10 | 5.7 | 8.3 | 3,800 |
| Comparative | Compound 52 | 10 | 6.5 | 6.2 | 2,500 |
| Example 1 | | | | | |
| Comparative | Compound 53 | 10 | 6.8 | 6.0 | 2,300 |
| Example 2 | | | | | |
| Comparative | Compound 54 | 10 | 5.5 | 8.1 | 1,300 |
| Example 3 | | | | | |
| Comparative | Compound 55 | 10 | 6.6 | 7.8 | 1,000 |
| Example 4 | | | | | |
| Comparative | Compound 56 | 10 | 6.8 | 7.2 | 1,800 |
| Example 5 | | | | | |
| Comparative | Compound 57 | 10 | 6.3 | 7.9 | 1,300 |
| Example 6 | | | | | |

As may be seen in Table 1, organic EL devices according to Examples 1 and 3 exhibited longer life, when compared to that of the Comparative Examples. Organic EL devices according to Examples 4 and 10 exhibited further longer life, when compared to that of the Comparative Examples. The organic EL devices according to the Examples exhibited higher efficiency than that of Comparative Examples 1 and 2. In addition, the organic EL devices according to Examples 4 and 5 were driven at a lower voltage than those of other Examples and the Comparative Examples.

For example, the lowering of a HOMO level, the lowering of a voltage and the high efficiency may be realized by introducing an aryl group between two carbazole groups in the compounds of the Examples. In addition, the carbazole group and the dibenzofuranyl moiety had high electron tolerance, and the compounds including the carbazole group or the dibenzofuranyl moiety exhibited longer life, when compared to that of a carbazole derivative not including the dibenzofuranyl moiety or an amine compound. For example, radical cleavage (due to a nitrogen atom and an oxygen atom) may be restrained, and long life may be realized by the introduction of position 3 or position 4 of the dibenzofuranyl moiety.

In the compounds according to the Examples, in which a phenylene group was connected between the carbazole group and the dibenzofuranyl moiety, the life was increased, when compared to the compounds according to the other Examples, in which the phenylene group was not introduced. For example, radicals may be stabilized through the increase of the conjugation to the nitrogen in the carbazole group by the phenylene group. The organic EL devices of Examples 4 and 5 included compounds having higher planarity than other compounds, the mobility of holes was improved, and the lowering of the voltage was attained. In addition, in the organic EL devices of Examples 13 and 14, planarity was decreased due to a naphthalenyl group, and the driving at a high voltage was induced. However, relatively high efficiency and long life were realized.

By way of summation and review, in the application of the organic EL device to a display apparatus, high efficiency and long life of the organic EL device may be desirable. For example, the emitting efficiency of the organic EL device in the blue emitting region may be lower than that in the red emitting region and the green emitting region, and the normalization, stabilization, etc. of a hole transport layer to realize the high efficiency and long life of the organic EL device may be considered.

The embodiments may provide a compound for an organic EL device having high efficiency and long life.

The compound for an organic EL device according to an embodiment may exhibit hole transport properties. The hole transport properties and electron tolerance of the organic EL device may be improved by introducing a dibenzofuranyl moiety or a dibenzothiophenyl moiety as an electron accepting substituent on the nitrogen atom of a heat resistant carbazole group, and the long life and high efficiency of the organic EL device may be realized. For example, marked effects may be obtained in a blue emitting region.

The compound for an organic EL device according to an embodiment may exhibit hole transport properties and may also have two strongly heat resistant carbazole groups. The hole transport properties and electron tolerance of the organic EL device may be improved by introducing a dibenzofuranyl moiety or a dibenzothiophenyl moiety as an electron accepting substituent on the nitrogen atom of one heat resistant carbazole group, and the long life and high efficiency of the organic EL device may be realized. For example, marked effects may be obtained in a blue emitting region.

In addition, the nitrogen atom of the carbazole group and the dibenzofuranyl moiety or the dibenzothiophenyl moiety may be connected via a phenylene group, radical reactivity of the nitrogen atom with an oxygen atom or a sulfur atom may be restrained through the conjugation of a benzene ring, and long life may be realized. For example, marked effects may be obtained in a blue emitting region.

In the compound for an organic EL device according to an embodiment, the dibenzofuranyl moiety or the dibenzothiophenyl moiety may be connected to the carbazole group at a position 2, position 3, or position 4 (of the dibenzofuranyl moiety or the dibenzothiophenyl moiety) via a divalent connecting group or a single bond, and the long life and high efficiency of the organic EL device may be realized.

According to an embodiment, a compound for an organic EL device and an organic EL device using the same, which may be driven at a low voltage, and have high efficiency and long life may be provided. For example, a compound for an organic EL device with high efficiency and long life in a blue emitting region, used as a layer among stacking layers disposed between a light emitting layer and an anode, and an organic EL device using the same may be provided. According to an embodiment, an organic EL device having improved hole transport properties and electron tolerance, long life and high efficiency in a blue emitting region may be realized by introducing a dibenzofuranyl moiety or a dibenzothiophenyl moiety as an electron accepting substituent on a nitrogen atom of a heat resistant carbazole group.

The compound for an organic EL device according to an embodiment may exhibit hole transport properties and may have two strongly heat resistant carbazole groups. Hole transport properties and electron tolerance may be improved by introducing a dibenzofuranyl moiety or a dibenzothiophenyl moiety as an electron accepting substituent in the nitrogen atom of one carbazole group. In addition, through combining the nitrogen atom of the carbazole group with the dibenzofuranyl moiety or the dibenzothiophenyl moiety via the phenylene group, radical reactivity due to the nitrogen atom with the oxygen atom or the sulfur atom may be restrained through the conjugation of a benzene ring, thereby realizing long life.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic electroluminescence (EL) device, the compound being represented by the following Formula (1): 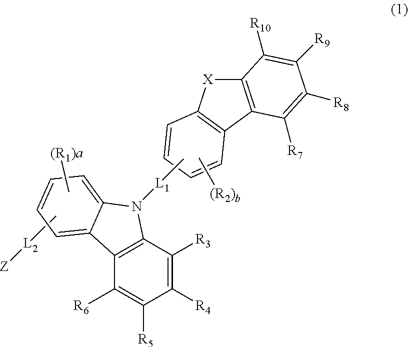
wherein, in Formula 1,
X is an oxygen atom or a sulfur atom,
R₁-R₁₀ are each independently an aryl group having 6 to 18 ring carbon atoms, a heteroaryl group having 5 to 18 ring carbon atoms, an alkyl group having 1 to 15 carbon atoms, a silyl group, a halogen atom, a hydrogen atom or a deuterium atom,
a and b are each independently an integer of 0 to 3,
L₁ is a divalent connecting group or a single bond,
L₂ is a divalent connecting group, and
Z is an aryl group having 6 to 30 ring carbon atoms or a heteroaryl group having 5 to 30 ring carbon atoms, wherein, when L₁ is the single bond, none of R₇-R₁₀ are a heteroaryl group.

2. The material for an organic EL device as claimed in claim 1, wherein at least two adjacent ones of R₁-R₁₀ are combined to each other and form a saturated or unsaturated ring, provided that an aromatic ring is not formed by a combination of R₁ with R₃ and R₆, or R₂ with R₇ and R₁₀.

3. The material for an organic EL device as claimed in claim 1, wherein a dibenzofuranyl moiety or a dibenzothiophenyl moiety of Formula (1) is bound to L₁ at position 2, position 3, or position 4 of the dibenzofuranyl moiety or the dibenzothiophenyl moiety.

4. A compound for an organic electroluminescence (EL) device, the compound being represented by the following Formula (2): 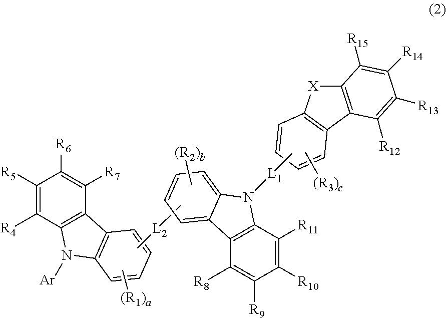
wherein, in Formula (2),
X is an oxygen atom or a sulfur atom,
Ar is an aryl group having 6 to 18 ring carbon atoms or an alkyl group having 1 to 15 carbon atoms,
R₁-R₁₅ are each independently an aryl group having 6 to 30 ring carbon atoms, an alkyl group having 1 to 15 carbon atoms, a silyl group, a halogen atom, a hydrogen atom or a deuterium atom,
a, b and c are each independently an integer of 0 to 3,
L₁ is a divalent connecting group or a single bond, and
L₂ is a divalent connecting group.

5. The material for an organic EL device as claimed in claim 4, wherein at least two adjacent ones of R₁-R₁₅ are combined to each other and form a saturated or unsaturated ring, provided that an aromatic ring is not formed by a combination of R₁ with R₄ and R₇, R₂ with R₈ and R₁₁, or R₃ with R₁₂ and R₁₅.

6. The material for an organic EL device as claimed in claim 4, wherein a dibenzofuranyl moiety or a dibenzothiophenyl moiety of Formula (2) is bound to L₁ at position 2, position 3, or position 4 of the dibenzofuranyl moiety or the dibenzothiophenyl moiety.

7. A compound for an organic electroluminescence (EL) device, the compound being represented by the following Formula (3): 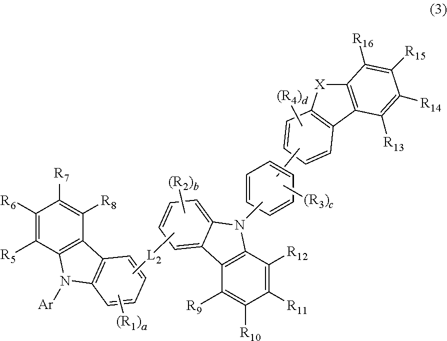
wherein, in Formula (3),
X is an oxygen atom or a sulfur atom,
Ar is an aryl group having 6 to 18 ring carbon atoms, a heteroaryl group having 5 to 18 ring carbon atoms, or an alkyl group having 1 to 15 carbon atoms,
R₁-R₁₆ are each independently an aryl group having 6 to 18 ring carbon atoms, a heteroaryl group having 5 to 18 ring carbon atoms, an alkyl group having 1 to 15 carbon atoms, a silyl group, a halogen atom, a hydrogen atom or a deuterium atom,
a, b, c and d are each independently an integer of 0 to 3, and
L₁ is a divalent connecting group.

8. The material for an organic EL device as claimed in claim 7, wherein Ar is an aryl group having 6 to 30 ring carbon atoms or an alkyl group having 1 to 15 carbon atoms.

9. The material for an organic EL device as claimed in claim 7, wherein at least two adjacent ones of R₁-R₁₆ are combined to each other and form a saturated or unsaturated ring, provided that an aromatic ring is not formed by a combination of R₁ with R₅ and R₈, R₂ with R₉ and R₁₂, or R₄ with R₁₃ and R₁₆.

10. The material for an organic EL device as claimed in claim 7, wherein a dibenzofuranyl moiety or a dibenzothiophenyl moiety of Formula (3) is bound to a phenylene moiety at position 2, position 3, or position 4 of the dibenzofuranyl moiety or the dibenzothiophenyl moiety.

11. An organic electroluminescence (EL) device, comprising:
an anode;
a cathode; and
a light emitting layer between the anode and the cathode,
wherein the light emitting layer includes the compound for an organic EL device as claimed in claim 10.

12. An organic electroluminescence (EL) device, comprising:
an anode;
a cathode;
a light emitting layer between the anode and the cathode; and
at least one stacking layer between the anode and the light emitting layer,
wherein the at least one stacking layer includes the compound for an organic EL device as claimed in claim 10.
